(19) United States
(12) Patent Application Publication
(10) Pub. No.: US 2025/0009505 A1
(43) Pub. Date: Jan. 9, 2025

(54) DELIVERY APPARATUS FOR MECHANICALLY EXPANDABLE VALVE

(71) Applicant: EDWARDS LIFESCIENCES CORPORATION, Irvine, CA (US)

(72) Inventor: Peleg Harel, Haifa (IL)

(21) Appl. No.: 18/893,663

(22) Filed: Sep. 23, 2024

Related U.S. Application Data
(63) Continuation of application No. PCT/US2023/016772, filed on Mar. 29, 2023.
(60) Provisional application No. 63/325,525, filed on Mar. 30, 2022.

(51) Int. Cl. A61F 2/24

(52) U.S. Cl. CPC A61F 2/243

(57) ABSTRACT

A delivery apparatus can include one or more actuator assemblies extending from a handle. Each actuator assembly can include an outer sleeve and an actuation member extending having an engagement portion configured to releasably couple an actuator of a prosthetic heart valve. The engagement portion can include a driver head comprising an engagement element configured to engage a corresponding engagement element in a head portion of the actuator, and a gripper member comprising a plurality of arms configured to releasably couple the head portion of the actuator. Each arm can have a distal end portion having a circumferential width wider than a circumferential width of a proximal end portion and an arcuate sectional shape. Rotation of the actuation member in a first direction can radially expands the prosthetic heart valve and rotation of the actuation member in a second direction can radially compress the prosthetic heart valve.

### CROSS REFERENCE TO RELATED APPLICATIONS

The present application is a continuation of International Application No. PCT/US2023/016772, filed on Mar. 29, 2023, which claims the benefit of U.S. Application No. 63/325,525, filed on Mar. 30, 2022, both of which are incorporated by reference herein in their entirety.

### FIELD

The present disclosure relates to implantable, mechanically expandable prosthetic devices, such as prosthetic heart valves, and to methods and delivery assemblies for, and including, such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed after an initial expansion (e.g., for repositioning and/or retrieval). However, some known devices and methods can cause rotation or movement of the prosthetic valve during expansion.

Despite the recent advancements in percutaneous valve technology, there remains a need for improved transcatheter heart valves and delivery devices for such valves.

### SUMMARY

Described herein are prosthetic heart valves, delivery apparatus, and methods for implanting prosthetic heart valves. The disclosed prosthetic heart valves, delivery apparatus, and methods can, for example, allow a delivery apparatus to radially expand a prosthetic heart valve at a selected implantation site. As such, the devices and methods disclosed herein can, among other things, overcome one or more of the deficiencies of typical prosthetic heart valves and their delivery apparatus.

A delivery apparatus for a prosthetic implant can comprise a handle and one or more shafts coupled to the handle.

In some examples, a delivery apparatus can comprise one or more actuator assemblies extending from the handle and configured to couple an actuate one or more actuators of a prosthetic heart valve in order to radially expand and radially compress the prosthetic heart valve.

In some examples, the actuator assemblies can each comprise a driver and an outer sleeve. In some examples, the driver can have an engagement portion comprising a driver head and a gripper member.

In some examples, the outer sleeve can comprise one or more support extensions extending partially over a radially inner or radially outer surface of the frame, the support extensions configured to inhibit rotation of the frame relative to the one or more actuator assemblies during expansion or compression of the prosthetic valve.

In some examples, the gripper member comprises a plurality of arms configured to releasably couple a head portion of the actuator of the prosthetic valve. In some examples, the distal end portion of each arm comprises a tooth extending radially inwardly, each tooth comprising an angled proximal surface. In some examples, each arm has a uniform thickness from a proximal end portion to a distal end portion of the arm.

In a representative example, delivery apparatus, can comprise one or more actuator assemblies extending from a handle. Each actuator assembly can comprise an outer sleeve and an actuation member extending through the outer sleeve and having a distal end portion configured to releasably couple an actuator of a prosthetic heart valve. The distal end portion can comprise a driver head comprising an engagement member extending distally from a main body of the driver head, the engagement member configured to extend into a corresponding recess in a head portion of the actuator, and a gripper member comprising a plurality of arms configured to releasably couple the head portion of the actuator. The plurality of arms can each comprise a distal end portion having a tooth extending radially inwardly toward a longitudinal axis of the actuation member and each arm can have an arcuate sectional shape. Rotation of the actuation member in a first direction radially expands the prosthetic heart valve and rotation of the actuation member in a second direction radially compresses the prosthetic heart valve.

In a representative example, an assembly can comprise a prosthetic heart valve and a delivery apparatus. The prosthetic heart valve can comprise a radially expandable and compressible frame having an inflow end portion and an outflow end portion, and one or more actuators. The actuators can have a main body and a head portion, the actuators configured to radially expand the frame upon rotation of the one or more actuators, and the head portion can comprise a central recess and a circumferentially extending recess. The delivery apparatus can comprise a handle, and one or more actuator assemblies extending from the handle. Each actuator assembly can include an outer sleeve and an actuation member extending through the outer sleeve, the actuation member having a distal end portion. The distal end portion can include a driver head comprising an engagement member extending distally from a main body of the driver head that extends into the central recess of a respective actuator, and a gripper member comprising a plurality of arms configured to releasably couple the head portion of the respective actuator, the plurality of arms each comprising a distal end portion having a tooth that extends into the circumferential recess of the respective actuator. Rotation of the actuation member in a first direction radially expands the prosthetic heart valve and rotation of the actuation member in a second direction radially compresses the prosthetic heart valve.

In a representative example, a method can comprise advancing an actuation member of an actuator assembly of a delivery apparatus axially relative to an actuator of a radially expandable and compressible prosthetic valve such that a plurality of arms of the actuation member advance over a head portion of the actuator, the head portion comprising a central recess and a circumferential recess. The method can further comprise continuing to advance the actuation member such that teeth extending radially inwardly from the plurality of arms arc disposed within the circumferential recess and such that an engagement member extending distally from the actuation member is disposed within the central recess, and advancing an outer sleeve of the actuator assembly over the actuation member such that the plurality of arms is radially compressed thereby retaining the teeth within the circumferential recess and coupling the actuator assembly to the prosthetic valve.

In another representative example, a delivery apparatus can comprise one or more actuator assemblies extending from a handle. Each actuator assembly can comprise an outer sleeve and an actuation member extending through the outer sleeve and having a distal end portion configured to releasably couple an actuator of a prosthetic heart valve. The distal end portion can include a driver head comprising an engagement member extending distally from a main body of the driver head, the engagement member configured to extend into a corresponding recess in a head portion of the actuator, the engagement member comprising one or more facets configured to engage corresponding facets in the corresponding recess, and a gripper member comprising a plurality of arms configured to releasably couple the head portion of the actuator, the plurality of arms each comprising a distal end portion having a tooth extending radially inwardly toward a longitudinal axis of the actuation member, the distal end portion of each arm having an increased circumferential width relative to a main body of the arm. Rotation of the actuation member in a first direction radially expands the prosthetic valve and rotation of the actuation member in a second direction radially compresses the prosthetic valve.

In a representative example, an implantable prosthetic device can comprise a radially expandable and compressible frame having an inflow end portion and an outflow end portion. The frame can comprise a plurality of posts, one or more of which are configured as actuation mechanisms including a first frame member having a first inner bore, a second frame member having a second inner bore, the first and second frame members being spaced apart axially from one another, an actuator, and a plurality of struts coupling adjacent posts to one another. The actuator can have an external threaded surface and can extend through the first and second inner bores. The actuator can further comprise a main body and a head portion, the head portion comprising a circumferential recess. Rotation of the actuator in a first direction results in axial movement of the first and second frame members toward one another to radially expand the prosthetic device.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side elevation view of a delivery apparatus for a prosthetic heart valve, according to one example.

FIG. 3 is a perspective view of a frame of a prosthetic heart valve comprising a plurality of actuator mechanisms one of which is shown coupled to an actuator assembly of a delivery apparatus, according to one example.

FIG. 4 is a perspective view of a portion of an actuator mechanism and actuator assembly of FIG. 3.

FIG. 5 is a perspective view of a portion of the actuator mechanism and actuator assembly of FIG. 4.

FIG. 6 is a perspective view of a portion of the actuator mechanism and actuator assembly of FIG. 4 with the outer sleeve shown transparently.

FIG. 7 is a perspective view of a portion of the actuator mechanism and actuator assembly of FIG. 4.

FIG. 8 is a perspective view of a portion of the frame and actuator assembly of FIG. 3 shown coupled together.

FIG. 9 is a perspective view of a portion of the frame and the actuator assembly of FIG. 3 shown coupled together with the outer sleeve shown transparently.

FIG. 10 is a perspective view of a portion of the frame and actuator assembly of FIG. 3 shown coupled together.

FIG. 11 is a partial cross-sectional view of a portion of the frame and actuator assembly of FIG. 3 shown coupled together.

FIG. 12 is a perspective view of a prosthetic heart valve, according to one example.

FIG. 13 is a perspective view of a portion of the frame of the prosthetic heart valve of FIG. 12.

FIG. 14 is a top plan schematic view of the prosthetic heart valve of FIG. 12.

FIG. 15 is a perspective view of a threaded rod of an actuator mechanism, according to one example.

FIG. 16 is a side elevation view of a portion of the threaded rod of FIG. 15.

FIG. 17 is an end-on view of the threaded rod of FIG. 15.

FIG. 18 is a perspective view of a portion of an outer sleeve of an actuator assembly, according to one example.

FIG. 19 is a perspective view of a portion of a driver of an actuator assembly, according to one example.

FIG. 20 is a perspective view of the driver head of the driver of FIG. 19.

FIG. 21 is a perspective view of the gripper member of the driver of FIG. 19.

FIGS. 22-25 illustrate perspective views of an exemplary method for decoupling an actuator assembly from an actuator mechanism of a prosthetic heart valve, according to one example.

FIGS. 26-28 illustrate cross-sectional side elevation views of the exemplary method for decoupling an actuator assembly of FIGS. 22-25.

FIG. 29 is a perspective view of a portion of an exemplary frame for a prosthetic heart valve coupled to an actuator assembly, according to one example.

FIGS. 30-32 illustrate perspective views of an exemplary method for decoupling the actuator assembly of FIG. 29 from an actuator mechanism of a prosthetic heart valve.

FIG. 33-35 illustrate cross-sectional views of the exemplary method for decoupling an actuator assembly of FIGS. 30-32.

FIG. 36 is a perspective view of a head portion of a threaded rod of a frame of a

prosthetic heart valve, according to one example.

FIG. 37 is a perspective view of a driver head of a driver of an actuator assembly, according to one example.

FIG. 38 is a perspective view of a gripper member of a driver of an actuator assembly, according to one example.

FIG. 39 is a perspective view of a portion of an exemplary frame for a prosthetic heart valve comprising the threaded rod of FIG. 36, and a portion of an actuator assembly comprising the driver head and gripper member of FIGS. 37-38, according to one example.

FIG. 40 is a perspective view of the actuator assembly of FIG. 39 coupled to a portion of the exemplary prosthetic heart valve frame of FIG. 39, according to one example.

FIG. 41 is a cross-sectional view of the actuator assembly of FIG. 39 coupled to a portion of the exemplary prosthetic heart valve frame of FIG. 39, according to one example.

FIG. 42 is a perspective view of a hypotube portion of an outer sleeve of an actuator assembly, according to one example.

FIG. 43 is a perspective view of an actuator assembly comprising the hypotube portion of FIG. 42 coupled to a portion of an exemplary prosthetic valve frame, according to one example.

FIG. 44 is a perspective view of the actuator assembly of FIG. 43 coupled to the prosthetic heart valve frame of FIG. 43, with the outer sleeve shown transparently for purposes of illustration.

FIG. 45 is a side elevation view of a portion of an outer sleeve of an actuation assembly comprising the hypotube portion of FIG. 42, showing the hypotube in an expanded configuration.

FIG. 46 is a side elevation view of the portion of the outer sleeve of FIG. 45, showing the hypotube in a compressed configuration.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

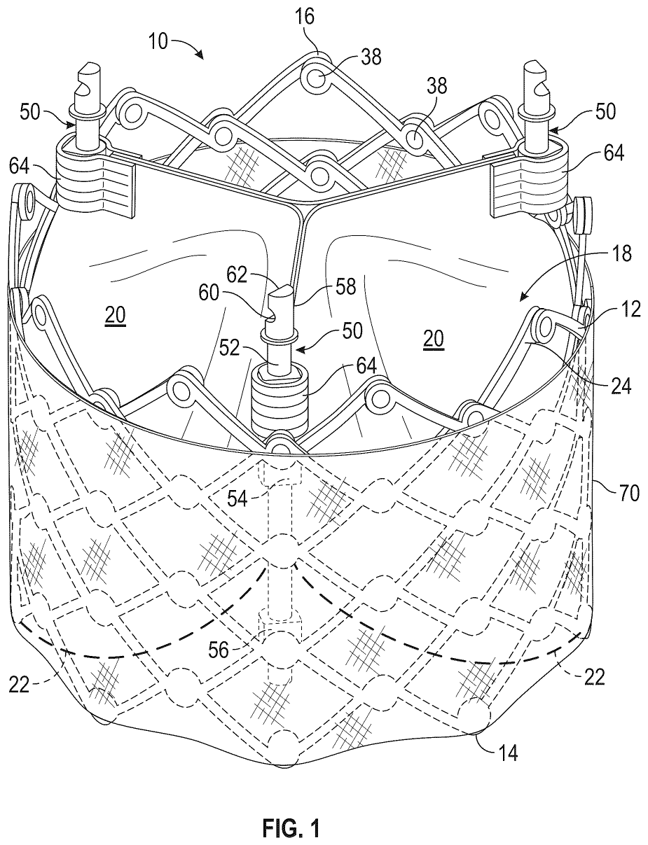

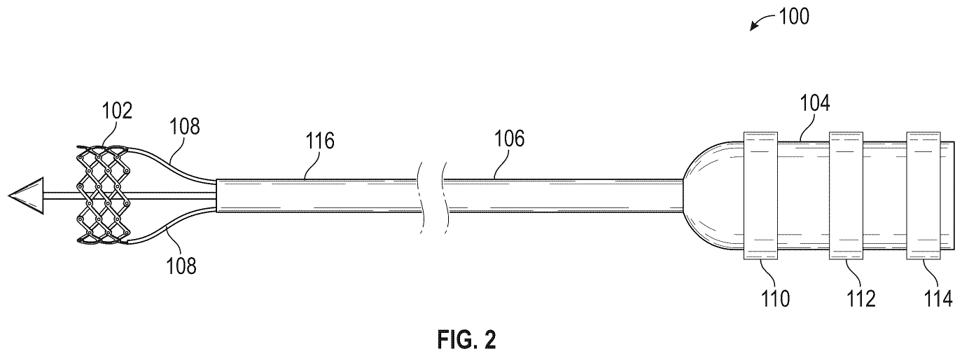

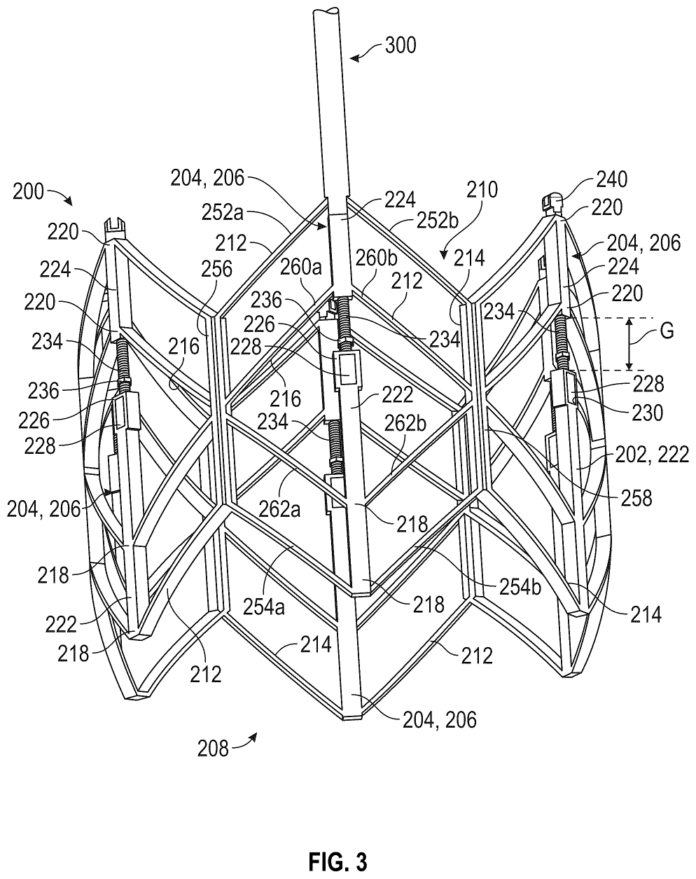

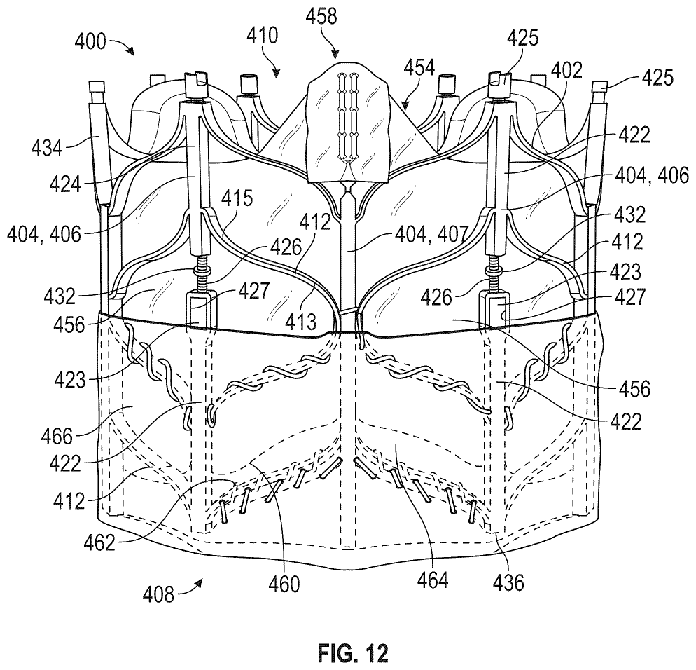

For example, a delivery apparatus 100 as shown in FIG. 2 can be used in combination with prosthetic valves 200 or 400 described herein. In another example, actuator mechanisms 206 as shown in FIG. 3 can be used in combination with the prosthetic valve 400 shown in FIG. 12 and/or prosthetic valve 10 shown in FIG. 1.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Examples of the Disclosed Technology

Described herein are examples of prosthetic implants, such as prosthetic heart valves, that can be implanted within any of the native valves of the heart (e.g., the aortic, mitral, tricuspid, and pulmonary valves). The present disclosure also provides frames for use with such prosthetic implants. The frames can further comprise actuator mechanisms (e.g., expansion mechanisms) and/or locking mechanisms to enable greater control over the radial compression or expansion of the valve body. The frames can comprise struts having different shapes and/or sizes to minimize the overall crimp profile of the implant and provide sufficient structural strength and rigidity to areas where needed.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the valves disclosed herein may be used with a variety of implant delivery apparatuses, and examples thereof will be discussed in more detail later.

FIG. 1 shows an exemplary prosthetic valve 10, according to one example. The prosthetic valve 10 can include an annular stent or frame 12 having an inflow end 14 and an outflow end 16. The prosthetic valve 10 can also include a valvular structure 18 which is coupled to and supported inside of the frame 12. The valvular structure 18 is configured to regulate the flow of blood through the prosthetic valve 10 from the inflow end 14 to the outflow end 16.

The valvular structure 18 can include, for example, a leaflet assembly comprising one or more leaflets 20 made of a flexible material. The leaflets 20 can be made from in whole or part, biological material, bio-compatible synthetic materials, or other such materials. Suitable biological material can include, for example, bovine pericardium (or pericardium from other sources). The leaflets 20 can be secured to one another at their adjacent sides to form commissures, each of which can be secured to a respective actuator mechanism 50 or the frame 12.

In the depicted example, the valvular structure 18 comprises three leaflets 20, which can be arranged to collapse in a tricuspid arrangement. Each leaflet 20 can have an inflow edge portion 22. As shown in FIG. 1, the inflow edge portions 22 of the leaflets 20 can define an undulating, curved scallop shape that follows or tracks a plurality of interconnected strut segments of the frame 12 in a circumferential direction when the frame 12 is in the radially expanded configuration. The inflow edges of the leaflets can be referred to as a “scallop line.”

In some examples, the inflow edge portions 22 of the leaflets 20 can be sutured to adjacent struts of the frame generally along the scallop line. In some examples, the inflow edge portions 22 of the leaflets 20 can be sutured to an inner skirt, which in turn in sutured to adjacent struts of the frame. By forming the leaflets 20 with this scallop geometry, stresses on the leaflets 20 are reduced, which in turn improves durability of the valve 10. Moreover, by virtue of the scallop shape, folds and ripples at the belly of each leaflet 20 (the central region of each leaflet), which can cause early calcification in those areas, can be eliminated or at least minimized. The scallop geometry also reduces the amount of tissue material used to form valvular structure 18, thereby allowing a smaller, more even crimped profile at the inflow end 14 of the valve 10.

Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, 8,252,202, and 11,135,056, and International Application No. PCT/US2020/024559, all of which are incorporated herein by reference in their entireties.

The prosthetic valve 10 can be radially compressible and expandable between a radially compressed configuration and a radially expanded configuration. The frame 12 can include a plurality of interconnected lattice struts 24 arranged in a lattice-type pattern and forming a plurality of apices 34 at the outflow end 16 of the prosthetic valve 10. The struts 24 can also form similar apices 32 at the inflow end 14 of the prosthetic valve 10.

The struts 24 can be pivotably coupled to one another at one or more pivot joints or pivot junctions 28 along the length of each strut. For example, in one example, each of the struts 24 can be formed with apertures 30 at opposing ends of the strut and apertures spaced along the length of the strut. Respective hinges can be formed at the locations where struts 24 overlap each other via fasteners 38, such as rivets or pins that extend through the apertures 30. The hinges can allow the struts 24 to pivot relative to one another as the frame 12 is radially expanded or compressed, such as during assembly, preparation, or implantation of the prosthetic valve 10.

The frame struts and the components used to form the pivot joints of the frame 12 (or any frames described below) can be made of any of various suitable materials, such as stainless steel, a cobalt chromium alloy, or a nickel titanium alloy (“NiTi”), for example Nitinol. In some examples, the frame 12 can be constructed by forming individual components (e.g., the struts and fasteners of the frame) and then mechanically assembling and connecting the individual components together. Further details regarding the construction of the frame and the prosthetic valve are described in U.S. Pat. Nos. 10,806,573, 10,603,165, 10,869,759, and 11,446,141, all of which are incorporated herein by reference in their entireties.

In the illustrated example, the prosthetic valve 10 can be mechanically expanded from the radially contracted configuration to the radially expanded configuration. For example, the prosthetic valve 10 can be radially expanded by maintaining the inflow end 14 of the frame 12 at a fixed position while applying a force in the axial direction against the outflow end 16 toward the inflow end 14. Alternatively, the prosthetic valve 10 can be expanded by applying an axial force against the inflow end 14 while maintaining the outflow end 16 at a fixed position, or by applying opposing axial forces to the inflow and outflow ends 14, 16, respectively.

As shown in FIG. 1, the prosthetic valve 10 can include one or more actuators 50 mounted to and equally spaced around the inner surface of the frame 12. Each of the actuators 50 can be configured to form a releasable connection with one or more respective actuators of a delivery apparatus.

In the illustrated example, expansion and compression forces can be applied to the frame by the actuators 50. Each of the actuators 50 can comprise a screw/actuation bolt/threaded rod 52, a first anchor in the form of a cylinder or sleeve 54, and a second anchor in the form of a threaded nut 56. The rod 52 extends through the sleeve 54 and the nut 56. The sleeve 54 can be secured to the frame 12, such as with a fastener 38 that forms a hinge at the junction between two struts. Each actuator 50 is configured to increase the distance between the attachment locations of a respective sleeve 54 and nut 56, which causes the frame 12 to elongate axially and compress radially, and to decrease the distance between the attachment locations of a respective sleeve 54 and nut 56, which causes the frame 12 to foreshorten axially and expand radially.

For example, each rod 52 can have external threads that engage internal threads of the nut 56 such that rotation of the rod causes corresponding axial movement of the nut 56 toward or away from the sleeve 54 (depending on the direction of rotation of the rod 52). This causes the hinges supporting the sleeve 54 and the nut 56 to move closer towards each other to radially expand the frame or to move farther away from each other to radially compress the frame, depending on the direction of rotation of the rod 52.

In some examples, the actuators 50 can be reciprocating type actuators configured to apply axial directed forces to the frame to produce radial expansion and compression of the frame. For example, the rod 52 of each actuator can be fixed axially relative to the nut 56 and slidable relative to the sleeve 54. Thus, in this manner, moving the rod 52 distally relative to the sleeve 54 and/or moving the sleeve 54 proximally relative to the rod 52 radially compresses the frame. Conversely, moving the rod 52 proximally relative to the sleeve 54 and/or moving the sleeve 54 distally relative to the rod 52 radially expands the frame.

When reciprocating type actuators are used, the prosthetic valve can also include one or more locking mechanisms that retain the frame in the expanded state. The locking mechanisms can be separate components that are mounted on the frame apart from the actuators, or they can be a sub-component of the actuators themselves.

Each rod 52 can include an attachment member 58 along a proximal end portion of the rod 52 configured to form a releasable connection with a corresponding actuator of a delivery apparatus. The actuator(s) of the delivery apparatus can apply forces to the rods for radially compressing or expanding the prosthetic valve 10. The attachment member 58 in the illustrated configuration comprises a notch 60 and a projection 62 that can engage a corresponding projection of an actuator of the delivery apparatus.

In the illustrated examples, the prosthetic valve 10 includes three such actuators 50, although a greater or fewer number of actuators could be used in other examples. The leaflets 20 can have commissure attachments members 64 that wrap around the sleeves 54 of the actuators 50. Further details of the actuators, locking mechanisms and delivery apparatuses for actuating the actuators can be found in U.S. Pat. Nos. 10,603,165 and 10,806,573, and 11,135,056, U.S. Publication No. 2022/0257367, and International Publication No. PCT/US2021/022467, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves discloses herein.

The prosthetic valve 10 can include one or more skirts or sealing members. In some examples, the prosthetic valve 10 can include an inner skirt (not shown) mounted on the inner surface of the frame. The inner skirt can function as a sealing member to prevent or decrease perivalvular leakage, to anchor the leaflets to the frame, and/or to protect the leaflets against damage caused by contact with the frame during crimping and during working cycles of the prosthetic valve. As shown in FIG. 1, the prosthetic valve 10 can also include an outer skirt 70 mounted on the outer surface of the frame 12. The outer skirt 70 can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Pat. No. 11,399,932, which is incorporated herein by reference in its entirety.

FIG. 2 illustrates a delivery apparatus 100, according to one example, adapted to deliver a prosthetic heart valve 102, such as the illustrated prosthetic heart valve 10, described above. The prosthetic valve 102 can be releasably coupled to the delivery apparatus 100. It should be understood that the delivery apparatus 100 and other delivery apparatuses disclosed herein can be used to implant prosthetic devices other than prosthetic valves, such as stents or grafts.

The delivery apparatus 100 in the illustrated example generally includes a handle 104, a first elongated shaft 106 (which comprises an outer shaft in the illustrated example) extending distally from the handle 104, at least one actuator assembly 108 extending distally through the outer shaft 106. The at least one actuator assembly 108 can be configured to radially expand and/or radially collapse the prosthetic valve 102 when actuated.

Though the illustrated example shows two actuator assemblies 108 for purposes of illustration, it should be understood that one actuator 108 can be provided for each actuator on the prosthetic valve. For example, three actuator assemblies 108 can be provided for a prosthetic valve having three actuators. In some examples, a greater or fewer number of actuator assemblies can be present.

In some examples, a distal end portion 116 of the shaft 106 can be sized to house the prosthetic valve in its radially compressed, delivery state during delivery of the prosthetic valve through the patient's vasculature. In this manner, the distal end portion 116 functions as a delivery sheath or capsule for the prosthetic valve during delivery,

The actuator assemblies 108 can be releasably coupled to the prosthetic valve 102. For example, in the illustrated example, each actuator assembly 108 can be coupled to a respective actuator of the prosthetic valve 102. Each actuator assembly 108 can comprise a support tube, an actuator member, and a locking tool. When actuated, the actuator assembly can transmit pushing and/or pulling forces to portions of the prosthetic valve to radially expand and collapse the prosthetic valve as previously described. The actuator assemblies 108 can be at least partially disposed radially within, and extend axially through, one or more lumens of the outer shaft 106. For example, the actuator assemblies 108 can extend through a central lumen of the shaft 106 or through separate respective lumens formed in the shaft 106.

The handle 104 of the delivery apparatus 100 can include one or more control mechanisms (e.g., knobs or other actuating mechanisms) for controlling different components of the delivery apparatus 100 in order to expand and/or deploy the prosthetic valve 102. For example, in the illustrated example the handle 104 comprises first, second, and third knobs 110, 112, and 114.

The first knob 110 can be a rotatable knob configured to produce axial movement of the outer shaft 106 relative to the prosthetic valve 102 in the distal and/or proximal directions in order to deploy the prosthetic valve from the delivery sheath 116 once the prosthetic valve has been advanced to a location at or adjacent the desired implantation location with the patient's body. For example, rotation of the first knob 110 in a first direction (e.g., clockwise) can retract the sheath 116 proximally relative to the prosthetic valve 102 and rotation of the first knob 110 in a second direction (e.g., counter-clockwise) can advance the sheath 116 distally. In some examples, the first knob 110 can be actuated by sliding or moving the knob 110 axially, such as pulling and/or pushing the knob. In some examples, actuation of the first knob 110 (rotation or sliding movement of the knob 110) can produce axial movement of the actuator assemblies 108 (and therefore the prosthetic valve 102) relative to the delivery sheath 116 to advance the prosthetic valve distally from the sheath 116.

The second knob 112 can be a rotatable knob configured to produce radial expansion and/or contraction of the prosthetic valve 102. For example, rotation of the second knob 112 can move the actuator member and the support tube axially relative to one another. Rotation of the second knob 112 in a first direction (e.g., clockwise) can radially expand the prosthetic valve 102 and rotation of the second knob 112 in a second direction (e.g., counter-clockwise) can radially collapse the prosthetic valve 102. In some examples, the second knob 112 can be actuated by sliding or moving the knob 112 axially, such as pulling and/or pushing the knob.

The third knob 114 can be a rotatable knob configured to retain the prosthetic heart valve 102 in its expanded configuration. For example, the third knob 114 can be operatively connected to a proximal end portion of the locking tool of each actuator assembly 108. Rotation of the third knob in a first direction (e.g., clockwise) can rotate each locking tool to advance the locking nuts to their distal positions to resist radial compression of the frame of the prosthetic valve, as described above. Rotation of the knob 114 in the opposite direction (e.g., counterclockwise) can rotate each locking tool in the opposite direction to decouple each locking tool from the prosthetic valve 102. In some examples, the third knob 114 can be actuated by sliding or moving the third knob 114 axially, such as pulling and/or pushing the knob.

Although not shown, the handle 104 can include a fourth rotatable knob operative connected to a proximal end portion of each actuator member. The fourth knob can be configured to rotate each actuator member, upon rotation of the knob, to unscrew each actuator member from the proximal portion of a respective actuator. As described above, once the locking tools and the actuator members are uncoupled from the prosthetic valve 102, they can be removed from the patient.

FIG. 3 illustrates an exemplary example of a prosthetic heart valve 200 comprising a unitary lattice frame 202. The prosthetic valve 200 can include a valvular structure comprising a plurality of leaflet (such as valvular structure 18 comprising leaflets 20) and inner and/or outer skirts, as previously described, though these components are omitted for purposes of illustration. The frame 202 can include one or more axially-extending struts or posts 204, one or more of which can be configured as integral expansion and locking mechanisms or actuator mechanisms 206 comprising an actuator 234 (such as a threaded rod) and first and second actuator struts/actuator posts 222, 224.

FIG. 3 illustrates the frame 202 in a partially expanded configuration and shows the frame 202 coupled to an actuator assembly 300 of a delivery apparatus (e.g., delivery apparatus 100).

The frame 202 can comprise an inflow end portion 208 (which is the distal end of the frame in the delivery configuration for the illustrated example) and an outflow end portion 210 (which is the proximal end portion of the frame in the delivery configuration for the illustrated example).

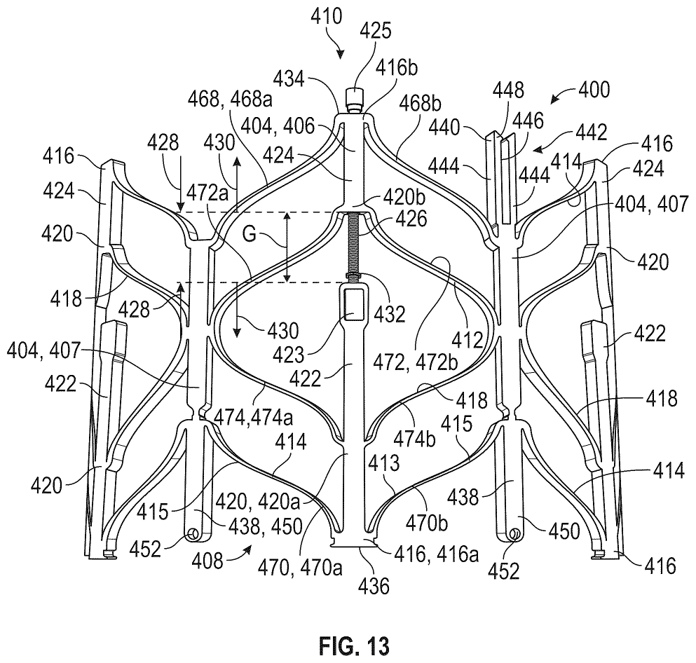

As mentioned, the frame 202 can comprise a plurality of axially-extending struts or posts 204, one or more of which can be configured as actuator mechanisms 206. The actuator mechanisms 206 can include a plurality of first actuator struts or actuator posts 222 (which are lower posts in the illustrated example and can extend to the inflow end of the frame) and a plurality of second actuator struts or actuator posts 224 (which are upper posts in the illustrated example and can extend to the outflow end of the frame). Each first actuator post 222 can be axially aligned with a corresponding second actuator post 224 to form a pair of first and second posts. One or more pairs of actuator posts 222, 224 can be configured as part of an actuator mechanism 206. The actuator posts 204 can be coupled together by a plurality of link members or struts 212. For example, in the illustrated example, the struts 212 define a plurality of cells extending circumferentially around the frame 202. The circumferentially-extending cells can include relatively larger hexagonal cells 214, and relatively smaller diamond-shaped cells 216 disposed within the hexagonal cells 214. However, in some examples, the cells can have any of various other shapes, for example, triangular, tear drop shaped, rectangular, square, oval, square-oval, etc. For example, as described in more detail below, FIG. 13 illustrates another example of a prosthetic heart valve 400 including a frame 402 having a plurality of axially-extending struts or posts 404 coupled together by a plurality of curved link members or struts 412.

Each hexagonal cell 214 is formed by two upper struts 252a, 252b, two lower struts 254a, 254b, and two axial struts 256, 258, each extending between and connecting respective ends of an upper strut 252a, 252b and a lower strut 254a, 254b. The upper struts 252a, 252b can be part of an upper row of struts that defines the outflow end of the frame and the lower struts 254a, 254b can be part of a lower row of struts that defines the inflow end of the frame.

Each diamond-shaped cell 216 is formed by two upper struts 260a, 260b and two lower struts 262a, 262b. The lower ends of the upper struts 260a, 260b and the upper ends of the lower struts 262a, 262b can be connected to the axial struts 256, 258. The upper ends of upper struts 252a, 252b of each hexagonal cell 214 can be connected to a second post 224 and the upper ends of upper struts 260a, 260b of the corresponding diamond-shaped cell 216 can be connected to the same second post 224. The lower ends of lower struts 254a, 254b of each hexagonal cell 214 can be connected to a first post 222 and the lower ends of lower struts 262a, 262b of the corresponding diamond-shaped cell 216 can be connected to the same first post 222.

In the illustrated example, there is one row of hexagonal cells 214 and one row of diamond-shaped cells 216. In alternative examples, the frame 202 can include a plurality of rows of hexagonal cells 214 and a plurality of rows of diamond-shaped cells 216 arrayed along the length of the frame, wherein the diamond-shaped cells 216 are positioned within the hexagonal cells.

The hexagonal and diamond cells 214, 216 can each comprise a respective inflow apex 218 and outflow apex 220. Each pair of actuator posts 222, 224 can extend through and be coupled to the inflow and outflow apices 218, 220 of a respective hexagonal cell 214 and diamond cell 216 pair. In the illustrated example, the frame 202 comprises six hexagonal cells 214 extending circumferentially in a row, with a diamond-shaped cell 216 within each hexagonal-shaped cell 214, and six pairs of actuator posts 222, 224 coupled to a respective pair of cells 214, 216. However, in some examples, the frame 202 can comprise a greater or fewer number of hexagonal cells 214 within a row, and a corresponding greater or fewer number of diamond cells 216, and/or pairs of actuator posts 222, 224.

In some examples, each pair of actuator posts 222, 224 can comprise part of an actuator mechanism 206 in combination with an actuator/threaded rod 234. For example, in the illustrated example, the frame comprises six pairs of posts 222, 224, each of which is configured as part of an actuator mechanism 206. In some examples, the frame 202 can comprise a greater or fewer number of actuator posts, and not all of the pairs of posts 222, 224 need to be actuator posts. Where a pair of posts 222, 224 is configured as part of an actuator mechanism, an actuator/screw/actuation bolt/threaded rod 234 extends through each actuator post 222, 224 of the pair to effect radially compression and expansion of the frame, as further described below. If a pair of posts 222, 224 is not configured as part of an actuator mechanism, an actuator/threaded rod 234 need not extend through the posts 222, 224 of that pair and the posts 222, 224 need not include the other features that are described below for radially compressing and expanding the frame.

Though in the illustrated example the actuator 234 is shown as a threaded rod, in some examples the actuator can be any of various members and/or mechanisms configured to move the first and second actuator posts 222, 224 axially relative to one another. For example, in some examples, the actuator 234 can be a linear rack comprising a plurality of teeth and configured to engage a corresponding pawl on the first and/or second actuator posts, or vice versa.

The upper end of each first actuator post 222 and the lower end of a corresponding second actuator post 224 can be separated by a gap G, allowing the actuator posts 222, 224 to move toward and away from each other during radial expansion and radial compression, respectively, of the frame. In the description that follows, the first and second actuator posts 222, 224, respectively, that are part of actuator mechanisms (i.e., those that include threaded rods 234), can also be referred to as first and second actuator frame members 222, 224, or more simply, first and second frame members 222, 224.

Each actuator frame member 222, 224 can comprise an inner bore 232 (FIG. 11) extending along a length of the frame member 222, 224 and through which a threaded rod 234 can extend. An outflow end portion 226 of the first frame member 222 can comprise or house a nut 228. In certain examples, the nut 228 can be separately formed from the frame member 222, which can be formed with a cut-out region defining window 230 in the outflow end portion of the frame member. The nut 228 can be positioned or housed within the cut-out region. As shown in FIG. 3, the nut 228 can be visible through the window 230. The nut 228 can comprise an inner threaded bore configured to engage the threads of the threaded rod 234 such that rotation of the threaded rod 234 causes the first frame member 222, which is coupled to the nut 228 to move relative to the second frame member 224, which is held steady.

In some examples, in lieu of using a nut 228, a portion of the inner bore 232 of the first frame member 222 can be threaded. For example, an outflow end portion of the first frame member 222 can comprise inner threads configured to engage the threaded rod 234 such that rotation of the threaded rod causes the first frame member 222 to move relative to the second frame member 224. In some examples, the inner bore 232 of the first frame member 222 may be threaded along its entire length. In alternative examples, each second frame member 224 can have internal threads or can house a nut 228 that can engage external threads of a threaded rod 234.

Rotation of the threaded rod 234 in a first direction (e.g., clockwise) causes corresponding axial movement of the first and second frame members 222, 224 toward one another, expanding the frame 202, and rotation of the threaded rod 234 in a second direction (e.g., counterclockwise) causes corresponding axial movement of the first and second frame members 222, 224 away from one another, compressing the frame. As the frame 202 moves from a compressed configuration to an expanded configuration, the gap G between the first and second frame members 222, 224 of the actuator mechanism 206 can narrow. The threaded rod 234 can comprise a stopper 236 (such as in the form of a nut) disposed thereon. As shown in FIG. 3, the stopper 236 can be disposed on the threaded rod 234 such that it sits within the gap G. During crimping/compression of the prosthetic valve 200, the threaded rod 234 can be rotated in the second direction (e.g., counterclockwise) causing the stopper 236 to move toward the outflow end portion 210 of the frame 202 until it abuts the inflow edge of the second frame member 224, thereby preventing over-crimping of the frame 202.

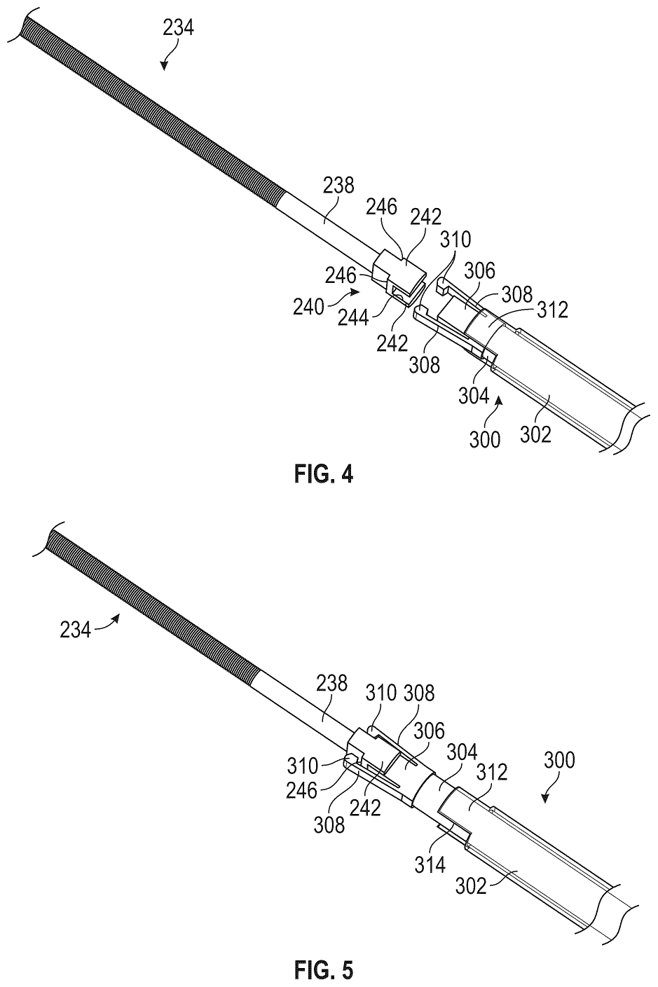

Referring to FIG. 4, the outflow or proximal end portion 238 of each threaded rod 234 can comprise a head portion 240 configured to be releasably coupled to a respective actuator assembly 300. The head portion 240 can comprise first and second protrusions 242 defining a channel or slot 244 between them, and one or more shoulders 246. As shown in FIG. 11, the head portion 240 can have a width greater than a diameter of the inner bore 232 such that the head portion 240 is prevented from moving into the inner bore 232 of the second frame member 224 and such that the head portion 240 abuts the outflow end portion 210 of the frame 402. The head portion 240 can be used to apply a distally-directed force to the second frame member 224, for example, during radial expansion of the frame 402.

Rotation of the threaded rod 234 while holding the second frame member 224 steady at a fixed location relative to the distal apparatus and the surrounding anatomy (e.g., using an actuator assembly 300 of the delivery apparatus) or applying a distally-directed force to the second frame member 224 causes axial movement of the inflow end 208 and outflow end 210 relative to one another to cause radial expansion or compression of the frame 202. For example, moving the inflow and outflow ends 208, 210 toward one another causes the frame to foreshorten axially and expand radially. Conversely, moving the inflow and outflow ends 208, 210 away from one another causes the frame 202 to elongate axially and compress radially.

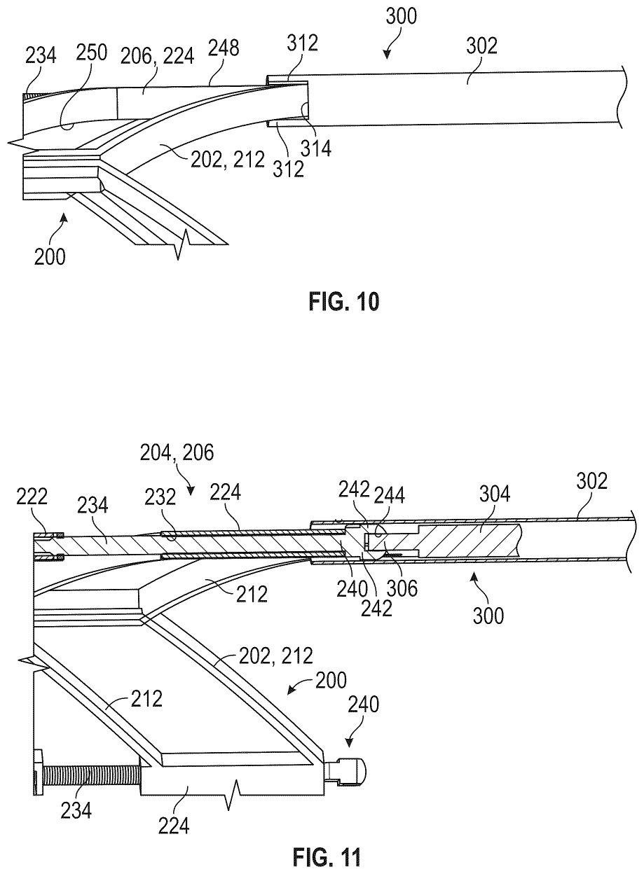

As shown in FIGS. 4-11, the threaded rod 234 of each actuator mechanism 206 can be releasably coupled to a respective actuator assembly 300 of a delivery apparatus, such as delivery apparatus 100, described previously. Referring to FIG. 4, each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-6 and 9 for purposes of illustration. The distal end portions of the outer sleeve 302 and driver 304 can be configured to engage or abut the proximal end (e.g., the outflow end) of the threaded rod 234 and/or the frame 202. The proximal portions of the outer sleeve 302 and driver 304 can be operatively coupled to the handle of a delivery apparatus (e.g., handle 104). The delivery apparatus in this example can include the same features described previously for delivery apparatus 100. In some examples, the proximal end portions of each driver 304 can be operatively connected to the knob 112 such that rotation of the knob 112 (clockwise or counterclockwise) causes corresponding rotation of the drivers 304. The proximal end portions of each outer sleeve 302 can be operatively connected to the knob 114 such that rotation of the knob 114 (clockwise or counterclockwise) causes corresponding axial movement of the sleeves 302 (proximally or distally) relative to the drivers 304. In some examples, the handle can include electric motors for actuating these components.

The distal end portion of the driver 304 can comprise a central protrusion 306 configured to extend into the slot 244 of the threaded rod 234, and one or more flexible elongated elements 308 including protrusions or teeth 310 configured to be releasably coupled to the shoulders 246 of the threaded rod 234. The protrusions 310 can extend radially inwardly toward a longitudinal axis of the driver/second actuation member 304. As shown in FIGS. 4-5, the elongated elements 308 can be configured to be biased radially outward to an expanded state, for example, by shape setting the elements 308.

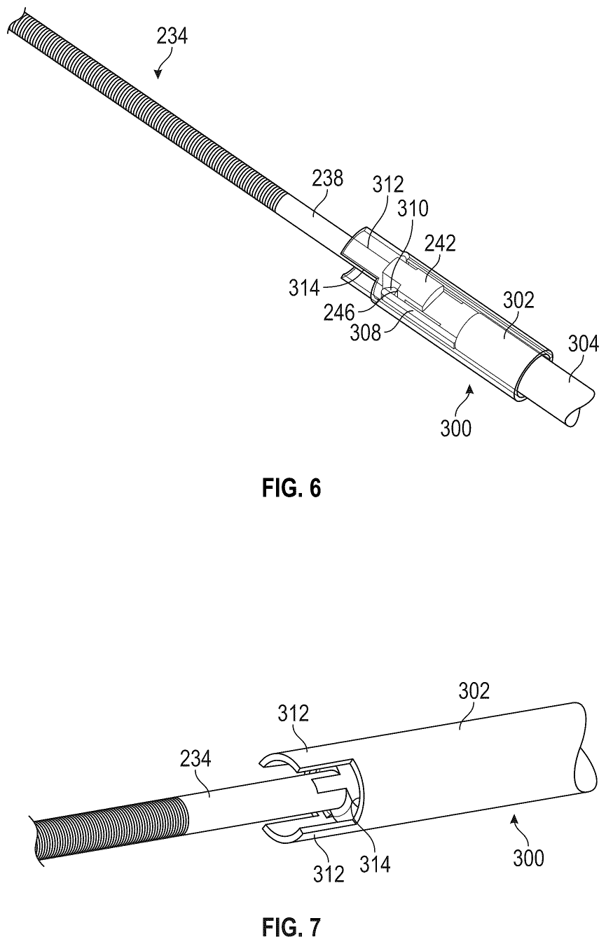

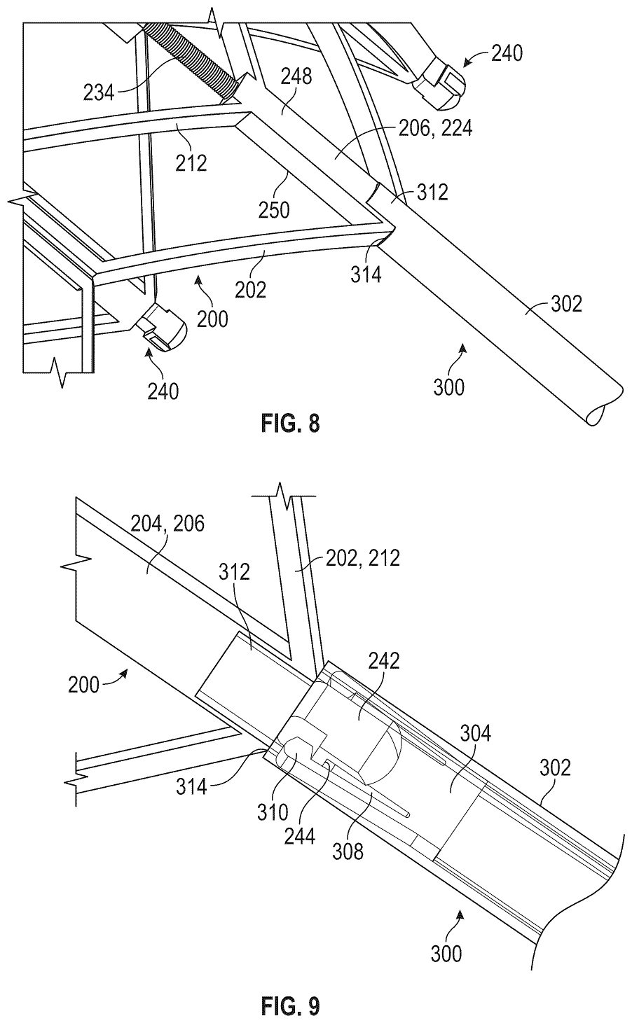

As shown in FIG. 5, to couple the actuator assembly 300 to the threaded rod 234, the driver 304 can be positioned such that the central protrusion 306 is disposed within the slot 244 (FIG. 4) and such that the protrusions 310 of the elongated elements 308 are positioned distally to the shoulders 246. Referring to FIG. 6, as the outer sleeve 302 is advanced (e.g., distally) over the driver 304, the elongated elements 308 are radially compressed until the protrusions 310 abut the shoulders 246, thereby coupling the actuator assembly 300 to the threaded rod 234. The outer sleeve 302 can continue to be advanced until the outer sleeve 302 engages the frame 202, as shown in FIG. 8. So coupled, the driver 304 can be rotated (e.g., using the handle of the delivery apparatus 100) to cause corresponding rotation of the threaded rod 234. The central protrusion 306 can be configured (e.g., sized and shaped) such that it is advantageously spaced apart from the inner walls of the outer sleeve 302, such that the central protrusion 306 does not frictionally contact the outer sleeve 302 during rotation.

Though in the illustrated example the central protrusion 306 has a substantially rectangular shape in cross-section, in some examples, the protrusion 306 can have any of various shapes, for example, square, triangular, oval, etc. The slot 244 can be correspondingly shaped to receive the protrusion 306.

Referring now to FIG. 7, the distal end portion of the outer sleeve 302 can comprise first and second support extensions 312 defining gaps or notches 314 between the extensions 312. As shown in FIG. 8, the support extensions 312 can be oriented such that, when the actuator assembly 300 is coupled to a respective actuator mechanism 206, the support extensions 312 extend partially over a proximal end portion (e.g., the upper end portion) of the second frame member 224, and in particular, a radially outer surface 248 and a radially inner surface 250 of the second frame member 224. The engagement of the support extensions 312 with the frame 202 counter-acts rotational forces applied to the frame by the rods 234 during expansion of the frame. In the absence of a counter-force acting against these rotational forces, the frame tends to “jerk” or rock in the direction of rotation of the rods when they are actuated to expand the frame. The illustrated configuration is advantageous in that outer sleeves, when engaging the second frame members 224, can prevent or mitigate such jerking or rocking motion of the frame when the frame is expanded.

A prosthetic valve 200 including one or more actuator mechanisms 206 can be expanded in the following exemplary manner. Generally, the prosthetic valve 200 is placed in a radially compressed state and releasably coupled to one or more actuator assemblies 300 of a delivery apparatus (such as delivery apparatus 100 shown in FIG. 2), as described above, and the delivery apparatus and the prosthetic valve can be advanced over a guidewire through the vasculature of a patient to a selected implantation site (e.g., the native aortic annulus). For example, when implanting the prosthetic valve within the native aortic valve, the delivery apparatus and the prosthetic valve can be inserted into and through a femoral artery, and through the aorta to the native aortic valve. The prosthetic valve 200 can then be deployed at the implantation site (e.g., within the native aortic valve) and can be expanded and locked in the expanded configuration using the actuator mechanisms 206. Once a selected diameter of the prosthetic valve 200 is reached, the actuator assemblies 300 can be uncoupled from the actuator mechanisms 206 and removed from the patient's body.

To deploy the prosthetic valve 200, the physician can actuate the actuator assemblies 300 by rotating the drivers 304 in a first direction (e.g., by rotating the knob 112 or actuating a motor), which can cause corresponding rotation of the threaded rods 234. The rotation of the threaded rod 234 can cause axial movement of the first and second frame members 222, 224 of the actuator mechanism 206 toward one another to decrease the distance between the frame members 222, 224, causing the frame 202 to foreshorten axially and expand radially until a selected diameter is achieved. The disclosed actuator mechanism examples advantageously allow for continuous prosthetic valve expansion (e.g., without the stepped expansion that results from a ratcheting mechanism) and allow the prosthetic heart valve to be deployed at any of various diameters.

Once the prosthetic valve 200 has been implanted at a selected implantation site within a patient, the patient's native anatomy (e.g., the native aortic annulus) may exert radial forces against the prosthetic valve 200 that would tend to compress the frame 202. However, the engagement of the threaded rod 234 with the threaded nut 228 prevents such forces from compressing the frame 202, thereby ensuring that the frame remains locked in the desired radially expanded state.

If repositioning or recapture and removal of the prosthetic valve 200 is desired, the prosthetic valve can be compressed (from an expanded or partially expanded configuration) by rotating the drivers 304 and therefore the threaded rods 234 in a second, opposing direction. The rotation of the threaded rods 234 can cause axial movement of the first and second frame members 222, 224 of the actuator mechanism 206 away from one another to increase the distance between the frame members 222, 224, causing the frame 202 to elongate axially and compress radially. Once the prosthetic valve 200 has been recompressed it can be repositioned at the implantation site, once repositioned, the prosthetic valve 200 can be expanded as described previously. The prosthetic valve can be re-compressed, repositioned, and re-expanded multiple times, as needed. In some cases, the prosthetic valve 200 can be fully compressed and “recaptured,” that is, retracted back into a sheath and/or removed from the patient's body.

Once final positioning and expansion of the prosthetic valve is achieved, the actuator assemblies 300 can be released from the prosthetic valve 200 by retracting the sleeves 302 to uncover the connection between the drivers 304 and the rods 234. This can be achieved by rotating the knob 114 or actuating a motor in the handle of the delivery apparatus. When each sleeve 302 is retracted, the expandable elements 308 of the driver 304 can expand outwardly and away from the shoulders 246 of the head portion 240 of the rod 234, thereby decoupling the driver 304 from the rod 234. At this stage, the delivery apparatus (including all of the actuator assemblies 300) can be retracted relative to the prosthetic valve 200 and removed from the patient's body.

FIG. 12 illustrates an example of a prosthetic valve 400 having a frame 402 including an inflow end portion 408 and an outflow end portion 410. The prosthetic valve 400 can further include a valvular structure 454, which is coupled to and supported inside the frame 402. The valvular structure 454 is configured to regulate the flow of blood through the prosthetic valve 400 from the inflow end 408 to the outflow end 410.

The prosthetic valve 400 can be similar to prosthetic valve 200, described above, including a frame 402 comprising a plurality of axially-extending posts 404, one or more of which (in combination with an actuator 426 such as a threaded rod) can be configured as actuator mechanisms 406, coupled together via a plurality of link members or struts 412. The prosthetic valve 400 in this example can include the same features described previously for prosthetic valve 200 and can be coupled to an actuator assembly 300 and expanded as described above for prosthetic valve 200.

The valvular structure 454 can include, for example, a leaflet assembly comprising one or more leaflets 456 made of flexible material. The leaflets 456 can be made from in whole or part, biological material, bio-compatible synthetic materials, or other such materials. Suitable biological material can include, for example, bovine pericardium (or pericardium from other sources). The leaflets 456 can be secured to one another at their adjacent sides to form commissures 458, each of which can be secured to a respective post 404 (e.g., to a support post 407) or to the frame 402.

In the depicted example, the valvular structure 454 includes three leaflets 456, which can be arranged to collapse in a tricuspid arrangement. Each leaflet 456 can have an inflow edge portion 460. As shown in FIG. 12, the inflow edge portions 460 of the leaflets 456 can define an undulating, curved scallop edge that follows or tracks portions of the struts 412 of frame 402 in a circumferential direction when the frame 402 is in the radially expanded configuration. The inflow edges 460 of the leaflets can be referred to as a “scallop line.”

As shown in FIG. 12, the inflow edge portions 460 of the leaflets 456 can be sutured to an inner skirt 464 generally along the scallop line. The inner skirt 464 can in turn be sutured to adjacent struts 412 of the frame 402, for example, via one or more sutures 462. In some examples, the leaflets 456 can be sutured directly to the frame 402 along the scallop line.

The prosthetic valve 400 can further include one or more skirts or scaling members. For example, as mentioned previously, the prosthetic valve 400 can comprise an inner skirt 464, mounted on the radially inner surface of the frame 402. The inner skirt 464 can function as a sealing member to prevent or decrease perivalvular leakage, to anchor the leaflets to the frame, and/or to protect the leaflets against damage caused by contact with the frame during crimping and during working cycles of the prosthetic valve. The prosthetic valve 400 can further include an outer skirt 466 mounted on the outer surface of the frame 402. The outer skirt 466 can function as a scaling member for the prosthetic valve by sealing against the tissue of the native valve annulus and helping to reduce paravalvular leakage past the prosthetic valve. The inner and outer skirts 464, 466 can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Patent Application No. 62/854,702.

The prosthetic valve 400 can be radially expandable and compressible between a radially expanded configuration and a radially compressed configuration. FIG. 13 shows the bare frame 402 of prosthetic valve 400 (without the leaflets or other components) for purposes of illustrating the configuration of the frame 402. While only one side of the frame 402 is depicted in FIG. 13, it should be appreciated that the frame 402 forms an annular structure having an opposite side that is substantially identical to the portion shown.

As shown in FIG. 13, the struts 412 of frame 402 can comprise a curved shape. The struts 412 can define a plurality of first and second cells extending circumferentially around the frame 402. Each first cell 414 can have an axially-extending elliptical shape including first and second apices 416 (e.g., inflow apex 416a and outflow apex 416b) disposed at the major vertices of the ellipse. Each first cell 414 can further comprise a respective second cell 418 disposed within the outer perimeter of the first cell 414. The second cell 418 can have a circumferentially-extending elliptical shape including first and second apices 420 (e.g., inflow apex 420a and outflow apex 420b) disposed at the minor vertices of the ellipse.

In some examples, such as the example shown in FIG. 13, each strut 412 can have a recurve or S-shape including a first, upwardly curved portion 413 and a second, downwardly curved portion 415 separated by an inflection point. Curved portion 413 has a convex curved surface facing the outflow end of the frame and a concave curved surface facing the inflow end of the frame, while curved portion 415 has a concave curved surface facing the outflow end of the frame and a convex curved surface facing the inflow end of the frame. Each strut 412 can terminate at either end in an asymptotic manner against a post 404 (e.g., a support post 407 and/or an actuator mechanism 406) such that it is nearly parallel with a longitudinal axis extending through the inflow and outflow ends of the frame 402. The struts 412 can be arranged such that the upwardly curved portion 413 is disposed nearer the inflow end 408 of the frame 402 and the downwardly curved portion 415 is disposed nearer the outflow end 410.

As mentioned, the frame 402 can comprise a plurality of axially-extending struts or posts 404, including a plurality of first actuator struts or actuator posts 422 (which are lower posts in the illustrated example and can extend to the inflow end of the frame) and a plurality of second actuator struts or actuator posts 424 (which are upper posts in the illustrated example and can extend to the outflow end of the frame). Each first actuator post 422 can be axially aligned with a corresponding second actuator post 424 for a pair of first and second actuator posts. One or more pairs of actuator posts 422, 424 in combination with an actuator 426 (such as a threaded rod) can be configured as actuator mechanisms 406. The frame 402 can further comprise additional posts 404 configured as support posts 407. The support posts 407 can be disposed between each pair of adjacent circumferentially disposed first cells 414, and the actuator mechanisms 406 can be disposed such that they extend through and are coupled to the apices 416, 420 of the first and second cells. The posts 404 can be coupled together via the struts 412.

Each first cell 414 is formed by two upper struts 468a, 468b and two lower struts 470a, 470b. Each upper and lower strut 468, 470 is coupled on one end to an actuator mechanism 406 and on the other end to a support post 407. The upper struts 468a, 468b can be part of an upper row of struts that defines the outflow end of the frame and the lower struts 470a, 470b can be part of a lower row of struts that defines the inflow end of the frame. Each second cell 418 is formed by two upper struts 472a, 472b and two lower struts 474a, 474b. The lower ends of the upper struts 472a, 472b and the upper ends of the lower struts 474a, 474b can be connected to the support posts 407. The upper ends of the upper struts 472a, 472b and the lower ends of the lower struts 474a, 474b can be connected to a respective actuator mechanism 406. In the illustrated example, the upper ends of the upper struts 472a, 472b can be connected to a second post 424 and the lower ends of the lower struts 474a, 474b can be connected to a first post 422.

As mentioned, the first and second cells 414, 418 can each comprise an inflow apex 416a, 420a and an outflow apex 416b, 420b. Each pair of posts 422, 424 can extend through and be coupled to the inflow and outflow apices 416, 420 of a respective first and second cell pair. In the illustrated example, the frame 402 comprises six first cells 414 extending circumferentially in a row, with a second cell 418 within each first cell 414, and six pairs of posts 422, 424 coupled to a respective pair of cells 414, 418. However, in some examples, the frame 402 can comprise a greater or fewer number of first cells 414 within a row, and a correspondingly greater or fewer number of second cells 418 and/or pairs of posts 422, 424.

In some examples, each pair of actuator posts 422, 424 can be configured as part of an actuator mechanism 406 in combination with an actuator 426 (such as a threaded rod). For example, in the illustrated example, each of the six pairs of actuator posts 422, 424 is configured as part of an actuator mechanism 406 in combination with an actuator 426. In some examples, not all pairs of actuator posts 422, 424 need be part of actuator mechanisms. Where a pair of actuator posts 422, 424 is configured as part of an actuator mechanism, a screw/actuator bolt/threaded rod 426 (including a head portion 425) extends through each actuator post 422, 424 of the pair to effect radial compression and expansion of the frame, similar to actuator mechanisms 206 described previously. If a pair of actuator posts 422, 424 is not used as an actuator mechanism, an actuator 426 need not extend through the actuator posts 422, 424 of that pair. In the description that follows, the first and second actuator posts 422, 424, respectively, that are used as actuator mechanisms (i.e., those that include actuators 426), can be referred to as first and second actuator frame members 422, 424, or more simply, first and second frame members 422, 424.

Though in the illustrated example the actuator 426 is shown as a threaded rod, in some examples the actuator 426 can be any of various members and/or mechanisms configured to move the first and second actuator posts 422, 424 axially relative to one another to expand and/or compress the frame 402. For example, in some examples, the actuator 426 can be a linear rack comprising a plurality of teeth and can be configured to engage a corresponding pawl on the first and/or second actuator posts, or vice versa.

In the illustrated example, the actuator mechanisms 406 can function in the same manner as actuator mechanisms 206 and can comprise a threaded nut 423 disposed at an outflow end portion of the first frame member 422 configured to engage the threaded rod 426. The nut 423 can be housed in a passageway of the second frame member 422 (e.g., disposed within a window 427). As shown in FIGS. 12-13, the nut 423 can be sized to fill the window 427 such that the nut 423 is restrained from movement relative to the second frame member 422.

Rotation of the threaded rod 426 in a first direction (e.g., clockwise) can cause corresponding axial movement of the first and second frame members 422, 424 toward one another (as shown by arrows 428), expanding the frame 402, and rotation of the threaded rod 426 in a second direction (e.g., counterclockwise) causes corresponding axial movement of the first and second frame members 422, 424 away from one another (as shown by arrows 430), compressing the frame. As the frame 402 moves from a compressed configuration to an expanded configuration, the gap G (FIG. 13) between the first and second frame members 422, 424 of the actuator mechanism 406 can narrow.

The threaded rod 426 can comprise a stopper 432 (e.g., a nut) disposed thereon. The stopper 432 can be disposed on the threaded rod 426 such that it sits within the gap G.

During crimping/compression of the prosthetic valve 400, the threaded rod 426 can be rotated in the second direction (e.g., counterclockwise) causing the stopper 432 to move toward the outflow end portion 434 of the frame 402 until it abuts the inflow edge of the second frame member 424.

Because the threaded rod 426 is secured to the frame 402 at axially spaced locations (the inflow end 408 and the outflow end 410) rotating the threaded rod 426 causes axial movement of the inflow end 436 and outflow end 434 relative to one another to cause radial expansion or compression of the frame 402. For example, moving the inflow and outflow ends 436, 434 toward one another causes the frame 402 to foreshorten axially and expand radially. Conversely, moving the inflow and outflow ends 436, 434 away from one another causes the frame 402 to elongate axially and compress radially.

As shown in FIG. 13, the support posts 407 can extend longitudinally and can have an inflow end portion 438 and an outflow end portion 440. The outflow end portion 440 of one or more support posts 407 can include a commissure support member 442. The commissure support member 442 can comprise first and second commissure arms 444 defining a commissure opening 446 between them. The commissure opening 446 can extend radially through a thickness of the post 404 and can be configured to accept a portion of the valvular structure 454 (e.g., the commissure 458) to couple the valvular structure 454 to the frame 402. In the illustrated example, the commissure opening 446 has a substantially rectangular shape and extends to the outflow end of the post 404. However, in some examples, the commissure opening can have any of various shapes (e.g., square, oval, square-oval, triangular, L-shaped, T-shaped, C-shaped, etc.). In some examples, the opening 446 can be fully enclosed by the post (e.g., not extending to the outflow edge) such that a portion of the valvular structure can be slid radially (rather than axially) into the commissure opening 446. The outflow end of each commissure arm 444 can include a tooth 448 extending into the commissure opening 446. The teeth 448 can help retain the commissure 458 within the commissure opening. Each commissure 458 can be mounted to a respective commissure support member 442, such as by inserting a pair of commissure tabs of adjacent leaflets through the opening 446 and suturing the commissure tabs to each other and/or the arms 444.

The inflow end portion 438 of each support post 407 can comprise a cantilevered extension 450 that extends toward the inflow end portion 436 of the frame 402. The extension 450 can comprise an aperture 452 extending radially through a thickness of the extension. In some examples, the extension 450 can extend such that an inflow edge of the extension aligns with or substantially aligns with an inflow edge of the frame 402. In use, the extension 450 can prevent or mitigate portions of the outer skirt 466 from extending radially inwardly and thereby prevent of mitigate any obstruction of flow through the inflow end 436 caused by the outer skirt 466. The extensions 450 can further serve as supports to which portions of the inner and/or outer skirts 464, 466 can be coupled. For example, sutures used to connect the inner and/or outer skirts can be wrapped around the extensions 450 and/or can extend through openings 452. In some examples, the cusp edge portions 460 of the leaflets 456 can be supported by the extensions 450, or selected ones of the extensions. For example, the cusp edge portions 460 can be secured to adjacent extensions 450 with sutures, which can extend through the openings 452.

As mentioned, FIG. 13 depicts only one side of the frame 402. Though only one support post 407 comprising a commissure support member 442 is shown in FIG. 13, it should be noted that the frame 402 can comprise any number of support posts 407, any number of which can be configured as commissure support members 442. For example, a frame 402 can comprise six support posts 407, three of which are configured as commissure support members 442. In some examples, for example, a frame can comprise one, two, three, or four commissure support members.

The actuator mechanisms 406 can be releasably coupled to one or more actuator assemblies 300, as described previously with respect to prosthetic valve 200. So coupled, the prosthetic heart valve 400 can be deployed at a selected implantation site using the same method described previously for prosthetic heart valve 200. The actuator assemblies 300 can actuate the actuator mechanisms 406 to expand and/or compress the frame 402.

Once the prosthetic valve 400 has been implanted at a selected implantation site within a patient, the patient's native anatomy (e.g., the native aortic annulus) may exert radial forces against the prosthetic valve 400 that would tend to compress the frame 402. However, the engagement of the threaded rod 426 with the threaded nut 423 of the first frame member 422 prevents such forces from compressing the frame 402, thereby ensuring that the frame remains locked in the desired radially expanded state.

If repositioning or recapture and removal of the prosthetic valve 400 is desired, the prosthetic valve can be compressed (from an expanded or partially expanded configuration) by rotating the threaded rod 426 (e.g., using driver 304 of actuator assembly 300) in a second, opposing direction. The rotation of the threaded rod 426 can cause axial movement of the first and second frame members 422, 424 of the post away from one another to increase the distance between the frame members 422, 424, causing the frame to elongate axially and compress radially. Once the prosthetic valve 400 has been recompressed it can be repositioned at the implantation site, once repositioned, the prosthetic valve 400 can be expanded as described previously. The prosthetic valve can be re-compressed, repositioned, and re-expanded multiple times, as needed. In some cases, the prosthetic valve 400 can be fully compressed and “recaptured,” that is, retracted back into a sheath and/or removed from the patient's body.

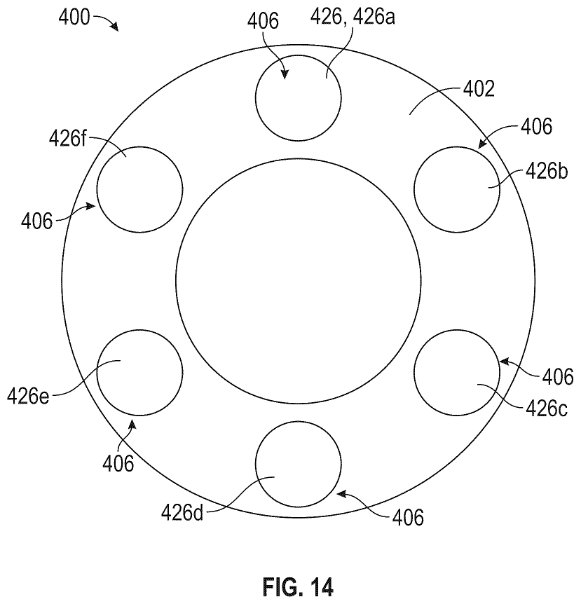

In some examples, selected threaded rods 426 can be configured as “right-hand” rods and selected threaded rods 426 can be configured as “left-hand” rods. As used herein, the term “right-hand” rod means a rod wherein rotation of the rod in a first direction (e.g., clockwise) effects expansion of the frame 402 and the term “left-hand” rod means a rod wherein rotation of the rod in a second direction (e.g., counter clockwise) effects expansion of the frame 402. Referring to FIG. 14, the prosthetic valve 400 can comprise six actuator mechanisms 406, each including a threaded rod 426 (e.g., threaded rods 426a, 426b, 426c, 426d, 426c, and 426f).

In some examples, the right-handed and left-handed rods can be disposed in an alternating pattern around the circumference of the valve 400 such that rods 426a, 426c, and 426c are configured as right-handed rods, and rods 426b, 426d, and 426f are configured as left-handed rods. During expansion of the frame 402, the right-handed rods 426a, 426c, 426c can be rotated (e.g., using a mechanism in the handle of the delivery apparatus) in a first direction (e.g., clockwise) and the left-handed rods 426b, 426d, and 426f can be rotated in a second direction (e.g., counter clockwise) opposite the first direction. During compression of the frame 402, the right handed rods 426a, 426c, 426e can be rotated in the second direction (e.g., counterclockwise) and the left-handed rods 426b, 426d, 426f can be rotated in the first direction (e.g., clockwise). The application of opposingly-directed rotational forces to alternating rods 426 can help prevent or mitigate jerking or rocking motions of the frame while the frame is expanded or compressed.

In some examples, the right-handed rods can be disposed along a first semi-circular portion of the circumference of the frame 402 and the left-handed rods can be disposed along a second semi-circular portion of the circumference, such that rods 426a, 426b, and 426c are configured as right-handed rods and rods 426d, 426c, and 426f are configured as left-handed rods. During expansion of the frame 402 the right-handed rods 426a, 426b, 426c can be rotated in a first direction (e.g., clockwise) and the left-handed rods 426d, 426c, 426f can be rotated in a second, opposing direction (e.g., counter clockwise). During compression of the frame 402, the right handed rods can be rotated in the second direction (e.g., counterclockwise) and the left-handed rods can be rotated in the first direction (e.g., clockwise).

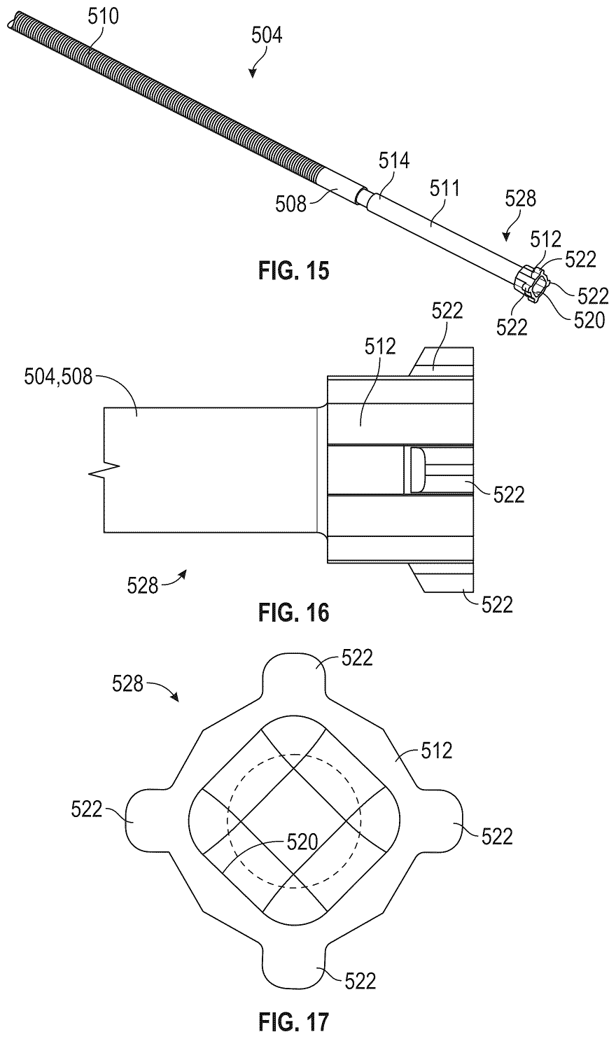

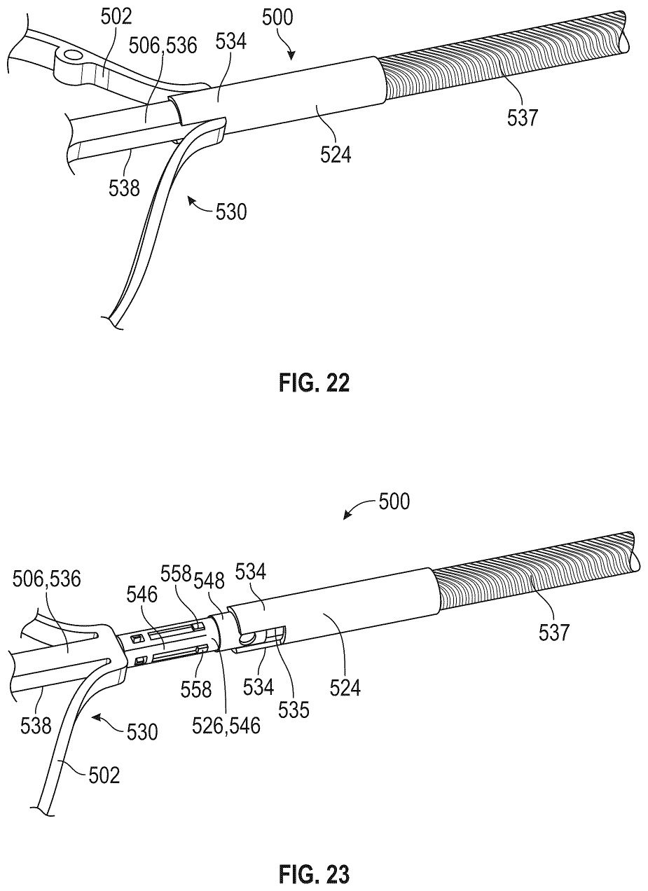

FIGS. 15-28 illustrate another example of an actuator assembly 500 (see e.g., FIG. 22) of a delivery apparatus that can be used to expand and/or compress a prosthetic valve, for example, any of the prosthetic valves disclosed herein. The actuator assembly 500 can be a component of a delivery apparatus (such as delivery apparatus 100, described previously), and can be configured to releasably couple and actuate a respective actuator/screw/actuation bolt/threaded rod of a prosthetic valve, such as a threaded rod 504 of actuator mechanism 506, partially shown in FIG. 22-25. Though not fully shown, actuator mechanism 506 can have first and second actuator posts/frame members separated by a gap through which the threaded rod 504 extends (similar to first and second frame members 422 and 424 of prosthetic valve 400). The threaded rod 504 can be used in lieu of or in addition to threaded rods 234 and/or 426 of prosthetic valves 200 and 400. The delivery apparatus can include a plurality of actuator assemblies 500, one for each threaded rod 504 of the prosthetic valve.

Referring to FIGS. 15-17, the threaded rod 504 can comprise an elongated body 508 having a threaded portion 510, a non-threaded portion 511, and a head portion 512 configured to be releasably coupled to a respective actuator assembly 500. In some examples, such as the illustrated example, the threaded rod 504 can comprise an annular notch 514 formed in the non-threaded portion 511. In some examples, the notch 514 can house a stopper, such as stopper 432 described previously, which can be swaged to the rod 504. In some examples, the threaded rod 504 can be formed without a notch 514. In such examples, the stopper can be welded to the rod 504 at a selected location.

As shown in FIG. 27, the head portion 512 can have a diameter DI greater than a diameter D₂ of the inner bore 516 of a frame 502 of a prosthetic valve, such that the head portion 512 is prevented from moving into the inner bore 516 of the frame 502 and such that the head portion 512 abuts the outflow edge 518 of the frame 502, e.g., as shown in FIG. 28. The head portion 512 of the threaded rod 504 can be used to apply a distally-directed force to the frame 502, for example, during radial expansion of the frame 502. The frame 502 can have the same configuration as frame 402.

The head portion 512 can comprise a central recess 520 and one or more projections 522 disposed about an outer perimeter of the head portion 512. In the illustrated example, as shown in FIG. 17, the projections 522 can comprise a substantially semi-circular shape in cross section. In some examples, the projections 522 can have any of various other shapes in cross section, such as square, rectangular, triangular, etc. Though the illustrated example shows four projections 522, in some examples the head portion 512 can have any number of projections, for example, one, two, three, four, five, six, seven, eight, etc. In the illustrated example, the central recess 520 is configured as a square recess with rounded corners, however, in some examples, the central recess 520 can have any of various other shapes, such as rectangular, ovular, triangular, etc.

Rotation of the threaded rod 504 while holding the outflow end of the frame 502 steady at a fixed location relative to the distal end portion of the delivery apparatus and the surrounding anatomy (e.g., using an actuator assembly 500 of the delivery apparatus) or applying a distally-directed force to the frame 502 causes axial movement of the inflow end and outflow end of the frame 502 relative to one another to cause radial expansion or compression of the frame. For example, moving the inflow and outflow ends toward one another causes the frame 502 to foreshorten axially and expand radially. Conversely, moving the inflow and outflow ends away from one another causes the frame 502 to elongate axially and compress radially.

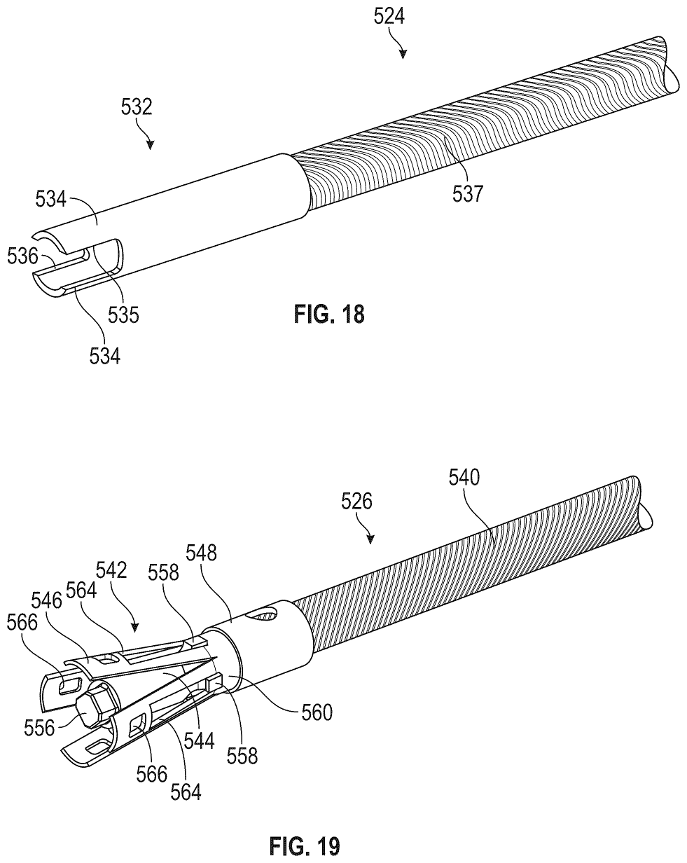

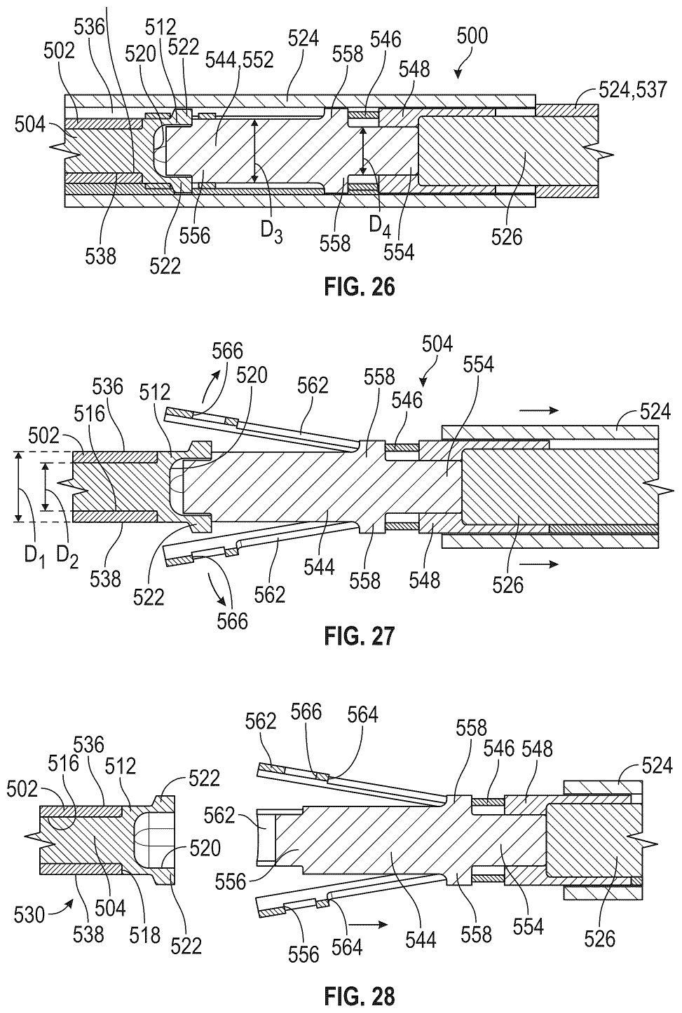

Referring to FIGS. 18-21, each actuation assembly 500 can comprise a first actuation member configured as a support tube or outer sleeve 524 (FIG. 18) and a second actuation member configured as a driver 526 (FIG. 19). As best seen in FIG. 26, the driver 526 can extend through the outer sleeve/support tube 524. The distal end portions of the outer sleeve 524 and driver 526 can be configured to engage or abut the outflow end portion 528 (FIG. 15) of the threaded rod 504 and/or the outflow end portion 530 of the frame 502. The proximal portions of the outer sleeve 524 and driver 526 can be operatively coupled to the handle of a delivery apparatus (e.g., handle 104 of delivery apparatus 100). The delivery apparatus in this example can include the same features described previously for delivery apparatus 100. In some examples, the proximal end portions of each driver 526 can be operatively connected to the knob 112 such that rotation of the knob 112 (clockwise or counterclockwise) causes corresponding rotation of the drivers 526. The proximal end portions of each outer sleeve 524 can be operatively connected to the knob 114 such that rotation of the knob 114 (clockwise or counterclockwise) causes corresponding axial movement of the sleeves 524 (proximally or distally) relative to the drivers 526. In some examples, the handle can include electric motors for actuating these components.

Referring to FIG. 18, a first or distal end portion 532 of the outer sleeve 524 can comprise first and second support extensions 534 defining gaps or notches 535 between them. As shown in FIG. 22, the support extensions 534 can be oriented such that, when the actuator assembly 500 is coupled to a respective actuator 506 of the frame 502, the support extensions 534 extend partially over an outflow end portion (e.g., the upper end portion) of the frame 502, such as an outflow apex or a strut portion, and in particular, over a radially outer surface 536 and a radially inner surface 538 of an outflow apex and a vertical strut extending from the apex. The engagement of the support extensions 534 with the frame 502 counter-acts rotational forces applied to the frame by the rods 504 during expansion of the frame 502. In the absence of a counter-force acting against these rotational forces, the frame tends to “jerk” or rock in the direction of rotation of the rods when they are actuated to expand the frame. The illustrated configuration is advantageous in that outer sleeves 524, when engaging the actuator mechanisms 506, can prevent or mitigate such jerking or rocking motion of the frame when the frame is expanded.

In some examples, as shown in FIG. 18, the distal end portion 532 of the outer sleeve 524 can comprise a different material and/or construction than the elongated body 537. For example, in some examples, the distal end portion 532 can be a laser cut tube formed comprising a nickel-cobalt base alloy, such as MP35N Alloy® and the elongated body 537 can be stainless steel. In some examples, the elongated body 537 can be a hollow, torque cable. In some examples, the elongated body 537 can be a laser cut slotted hypotube.

Referring to FIG. 19, the driver 526 can comprise an elongated body 540, and an engagement portion 542 disposed at a distal end portion of the elongated body 540. In some examples, the elongated body 540 can comprise a cable, for example, a 0.85 mm cable. In some examples, the elongated body 540 can be, for example, a slotted hypotube. The engagement portion 542 can comprise a driver head 544 and a gripper member 546. As shown in the illustrated example, the driver head 544 can be coupled to the elongated body using, for example, a fastener 548. In some examples, the driver head 544 can be welded or otherwise permanently coupled to the elongated body 540. In some examples, the driver head 544 and the elongated body 540 can be formed integrally with one another.

In some examples, the gripper member 546 can be coupled to the elongated body 540 by sandwiching the annular base 560 of the gripper member 546 between the second end portion 554 of the driver head 544 and the fastener 548. The fastener 548 can then be welded to the driver head 544, capturing the annular base 560 between them such that the gripper member is restrained from axial movement relative to the driver head 544. In some examples, the gripper member 546 can be formed integrally with the fastener 548, or otherwise can be coupled to the fastener 548 such as via welding. In some examples, the elongated body 540, driver head 544, and gripper member 546 can be welded together, while in some examples, they can be coupled together using a swaged coupling.

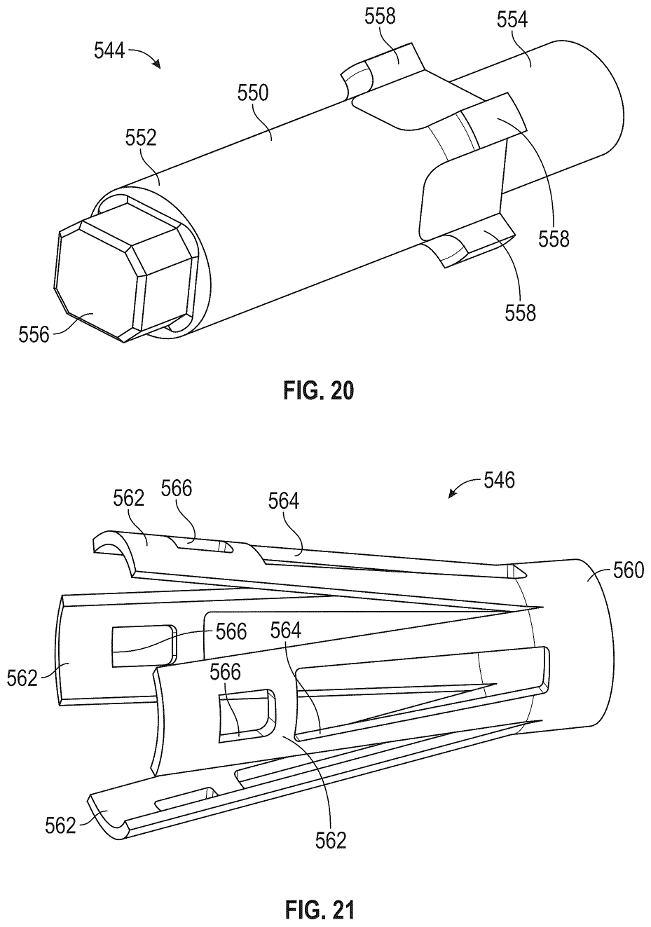

Referring to FIG. 20, the driver head 544 can comprise a main body 550 having a first end portion 552 and a second end portion 554, an engagement member 556, and one or more extension members/projections 558. The one or more projections 558 can extend radially outwardly from the main body 550 and can be spaced apart from one another around a perimeter of the driver head 544. The engagement member 556 can extend distally from the first end portion 552 of the main body 550 and can be configured to engage the recess 520 of a respective threaded rod 504. Though in the illustrated example the engagement member 556 has a square shape with chamfered edges in cross-section, in some examples, the engagement member 556 can have any of various shapes, for example, rectangular, triangular, ovular, etc. The recess 520 in the head portion 512 of the threaded rod 504 can be correspondingly shaped to receive the engagement member 556. The shape of the engagement member 556 and the corresponding recess 520 can advantageously improve torque transmission from the driver 526 to the threaded rod 504.

As best seen in FIG. 26, the first end portion 552 of the driver head 544 can have a first diameter D₃ and the second end portion 554 can have a second, smaller diameter D₄. The second end portion 554 can extend into the fastener 548 to couple the driver head 544 to the elongated body 540. In some examples, the driver head 544 can comprise a nickel-cobalt base alloy, such as MP35N Alloy®.

Referring now to FIG. 21, the gripper member 546 can comprise an annular base member 560 and one or more extension members/wings/arms 562. The arms 562 can extend distally from the base member 560 and can be resilient such that they are movable between an expanded position (FIG. 19) and a compressed position (FIG. 23). The arms 562 can be configured to bias radially outwardly (e.g., away from longitudinal axis of the driver 526) into the expanded shape, for example, by shape setting the arms 562. Each arm 562 can comprise a first, elongated opening 564 and a second opening 566. In the illustrated example, the gripper member 546 comprises four arms, however, in some examples the gripper member 546 can comprise any number of arms, for example, one, two, three, four, five, six, seven, eight, etc. In some examples, the gripper member 546 can comprise a laser-cut Nitinol tube, which can be heat-set such that the arms 562 bias radially outwardly.

As shown in FIG. 19, the projections 558 of the driver head 544 can extend into the first openings 564 to restrain the gripper member 546 against axial and rotational movement relative to the driver head 544. The second openings 566 can be configured (e.g., sized and shaped) to engage the projections 522 disposed on the head portion 512 of a respective threaded rod 504, thereby releasably coupling the actuation assembly 500 to the threaded rod 504. In the illustrated example, the first opening 564 is an elongated, rectangular opening and the second opening 566 is a substantially square opening, however, in some examples, the openings 564, 566 can have any of various shapes corresponding to the shape and size of the projections 558 of the driver head 544 and the projections 522 of the threaded rod 504, respectively.

The actuator assembly 500 can couple a respective actuator mechanism 506 of a prosthetic valve as follows. The engagement member 556 of the driver 526 can be disposed within the recess 520 of the head portion 512 of a threaded rod 504 such that the second openings 566 of the gripper member arms 562 are disposed adjacent the projections 522. As the outer sleeve 524 is advanced (e.g., distally) over the driver 526, the arms 562 are radially compressed until the projections 522 sit within the second openings 566, thereby coupling the actuator assembly 500 to the threaded rod 504 (e.g., as shown in FIG. 26). The outer sleeve 524 can continue to be advanced until the support extensions 534 engage the radially inner and outer surfaces 536, 538 of the frame 502, as shown in FIG. 22. So coupled, the driver 526 can be rotated (e.g., using the handle of the delivery apparatus 100) to cause corresponding rotation of the threaded rod 504. The engagement between the actuator assembly 500 and the threaded rod 504 can advantageously improve torque transfer between the driver and the threaded rod 504.

Generally, the prosthetic valve is placed in a radially compressed state and the actuator mechanisms 506 of the prosthetic valve are releasably coupled to one or more actuator assemblies 500 in the manner described previously, as shown in FIGS. 22 and 26. The delivery apparatus and the prosthetic valve can be advanced over a guidewire through the vasculature of a patient to a selected implantation site (e.g., the native aortic annulus). For example, when implanting the prosthetic valve within the native aortic valve, the delivery apparatus and the prosthetic valve can be inserted into and through a femoral artery, and through the aorta to the native aortic valve. The prosthetic valve can then be deployed at the implantation site (e.g., within the native aortic valve) and can be expanded and locked in the expanded configuration using the actuator mechanisms 506.

To deploy the prosthetic valve, the physician can actuate the actuator mechanisms 506 by rotating the drivers 526 in a first direction (e.g., by rotating the knob 112 or actuating a motor), which causes corresponding rotation of the threaded rods 504. The rotation of the threaded rod 504 in the first direction causes axial movement of the first and second frame members of the actuator mechanism (e.g., similar to first and second frame members 422, 424 of prosthetic valve 400) toward one another, causing the frame 502 to foreshorten axially and expand radially until a selected diameter is achieved. The disclosed actuator mechanism examples advantageously allow for continuous prosthetic valve expansion (e.g., without the stepped expansion that results from a ratcheting mechanism) and allow the prosthetic heart valve to be deployed at any of various diameters.

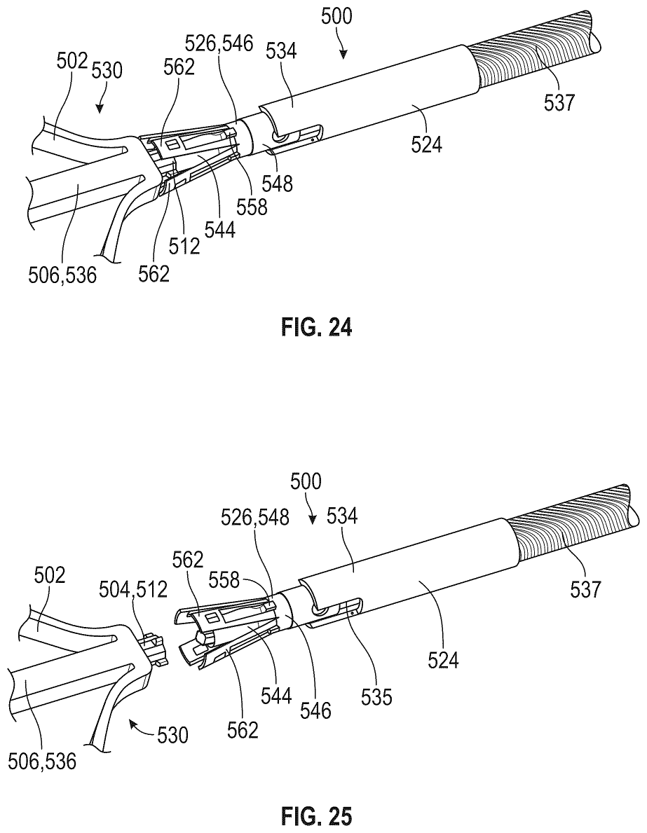

Referring to FIGS. 22-28, once final positioning and expansion of the prosthetic valve is achieved, the actuator assemblies 500 can be released from the prosthetic valve in the following exemplary manner. The outer sleeve 524 can be retracted to uncover the connection between the driver 526 and the threaded rod 504, as shown in FIGS. 23 and 27. This can be achieved by rotating the knob 114 or actuating a motor in the handle of the delivery apparatus 100. As shown in FIGS. 24 and 27, once the outer sleeve 524 is retracted, the arms 562 of the gripper member 546 can expand radially outwardly away from the head portion 512 of the threaded rod 504. As shown in FIGS. 25 and 28, the driver 526 can then be retracted, removing the engagement member 556 from within the recess 520 of the rod 504, thereby decoupling the driver 526 from the rod 504. At this stage, the delivery apparatus (including the actuator assemblies 500) can be retracted relative to the prosthetic valve and removed from the patient's body.

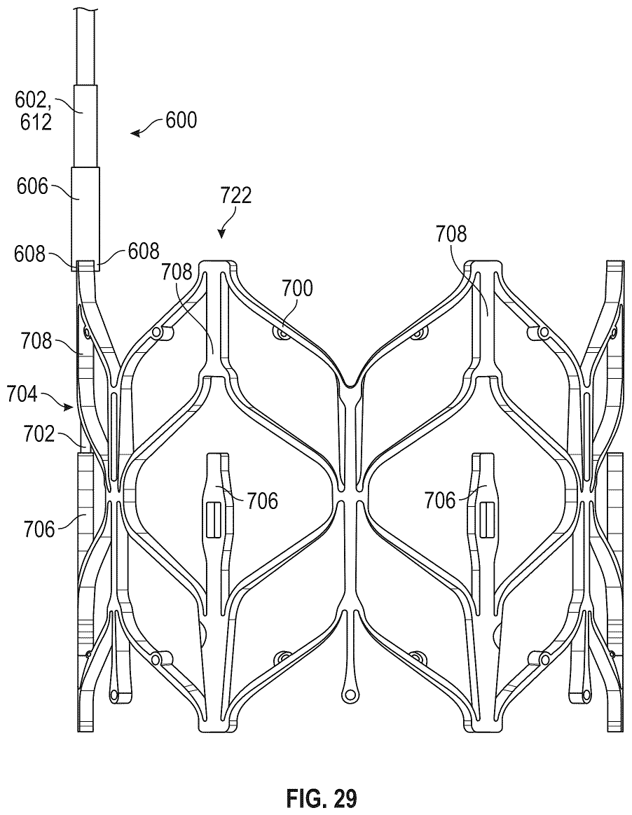

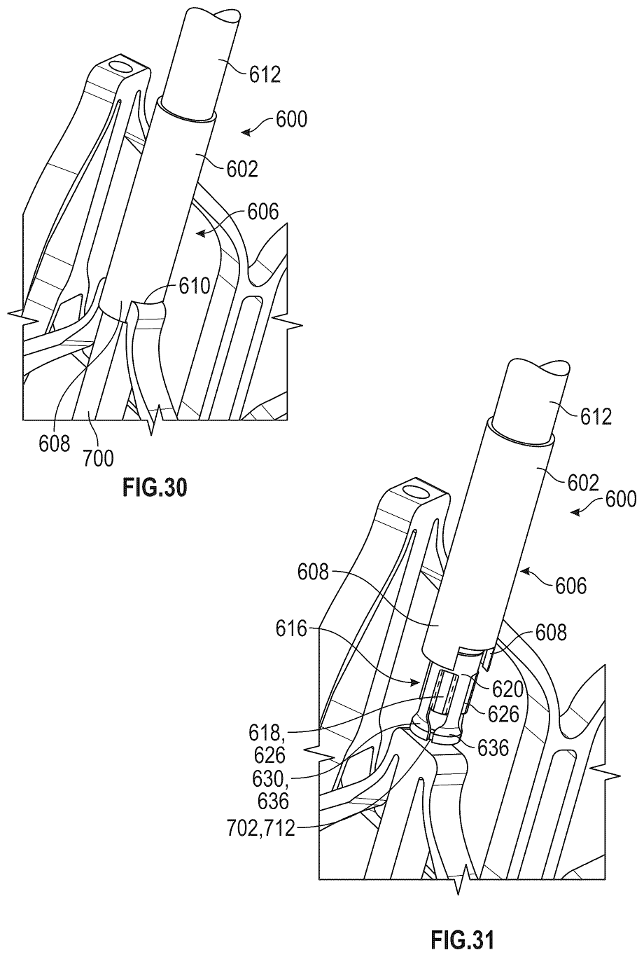

FIGS. 29-35 illustrate another example of an actuator assembly 600 of a delivery apparatus that can be used to radially expand and/or compress a prosthetic valve. FIG. 29 illustrates the actuator assembly 600 being used on an exemplary frame 700 of a prosthetic valve, however, the actuator assembly 600 can be used on any of the prosthetic valves described herein and/or other prosthetic valves having similar expansion mechanisms.

Though only one side of frame 700 is shown in FIG. 29, it should be appreciated that frame 700 forms an annular structure having an opposite side that is substantially identical to the one shown. Further, though not shown, frame 700 can support a valvular structure and can comprise inner and/or outer skirts to form a prosthetic heart valve similar to prosthetic heart valve 400.

The actuator assembly 600 can be a component of a delivery apparatus (such as delivery apparatus 100, described previously), and can be configured to releasably couple and actuate a respective actuator/screw/actuation bolt/threaded rod of a prosthetic valve, such as threaded rod 702 of actuator mechanism 704, partially shown in FIG. 29-35. Referring to FIG. 29, actuator mechanism 704 can have first and second actuator posts/frame members 706, 708 separated by a gap through which the threaded rod 702 extends (similar to first and second frame members 422 and 424 of prosthetic valve 400). The threaded rod 702 can be used in lieu of or in addition to threaded rods 234 and/or 426 of prosthetic valves 200 and 400. The delivery apparatus can include a plurality of actuator assemblies 600, one for each threaded rod 702 of the prosthetic valve. The delivery apparatus can include any number of actuator assemblies (e.g., 1-15 actuator assemblies). The number of actuation assemblies of the delivery apparatus can, for example, be determined by the number of threaded rods of the prosthetic valve for which the delivery apparatus is being used to implant. For example, a delivery apparatus comprising three actuator assemblies can be used with a prosthetic valve comprising three threaded rods. As another example, a delivery apparatus comprising six actuator assemblies can be used with a prosthetic valve comprising six threaded rods. Though only one threaded rod 702 is shown in FIG. 29 for purposes of illustration, it should be understood that each pair of first and second actuator posts/frame members can comprise a threaded rod 702.

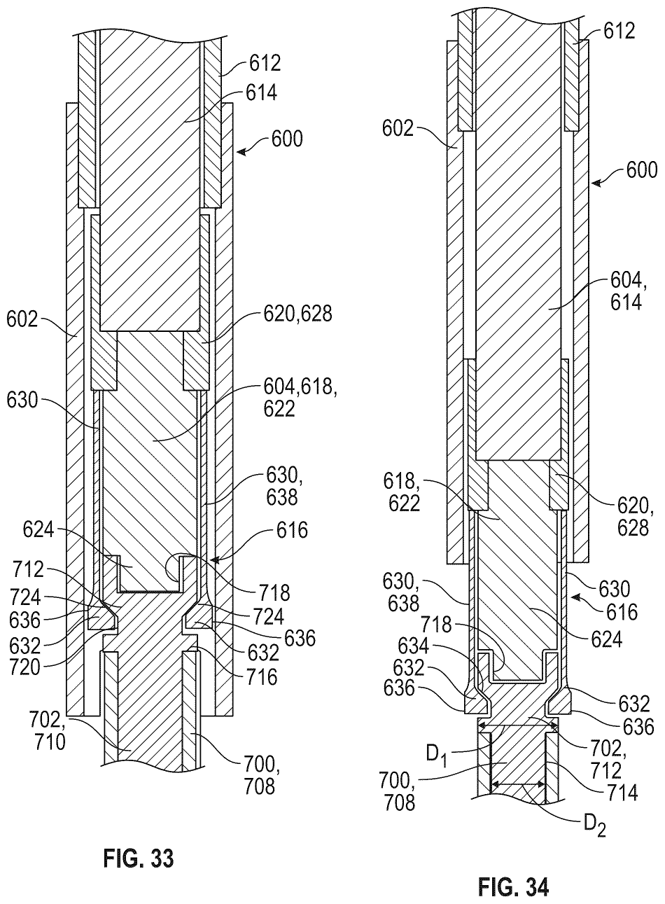

The threaded rod 702 can comprise an elongated body 710 (partially shown in FIG. 33) and a head portion 712 configured to be releasably coupled to a respective actuator assembly 600. As shown in FIG. 34, the head portion 712 can have a diameter Di greater than a diameter D₂ of the inner bore 714 of the second post 708, such that the head portion 712 is prevented from moving into the inner bore 714 of the frame 700 and such that the head portion 712 abuts the outflow edge 716 of the frame 700, e.g., as shown in FIG. 34. The head portion 712 of the threaded rod 702 can be used to apply a distally-directed force to the frame 700, for example, during radial expansion of the frame 700. Frame 700 can be similar to frame 402 of prosthetic heart valve 400.

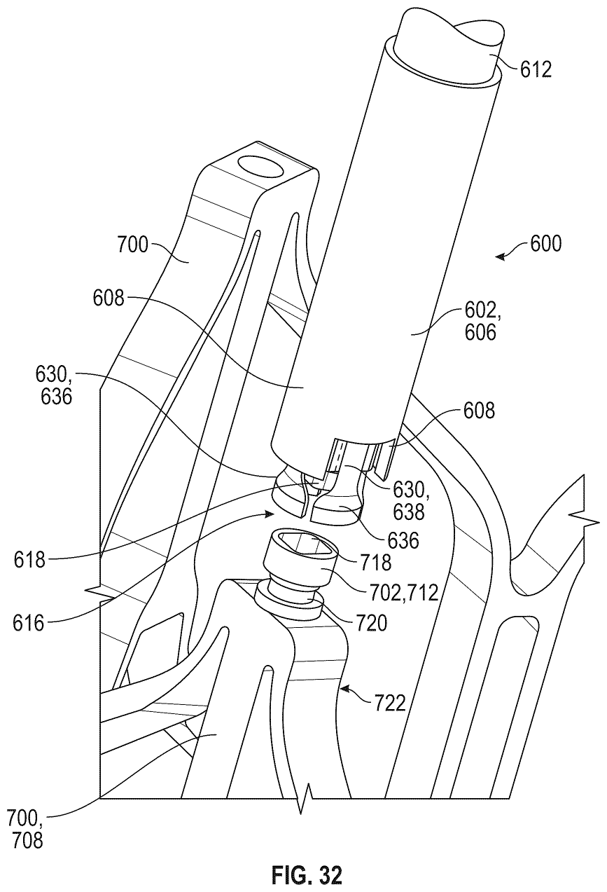

Referring to FIG. 32, the head portion 712 can comprise a central recess 718 and a circumferential recess 720 extending around the head portion 712. In the illustrated example, the central recess 718 is configured as a square recess, however, in some examples, the central recess can have rounded corner (e.g., similar to recess 520) or can have any of various other non-circular shapes, such as rectangular, ovular, triangular, star, etc. In some examples, the central recess 718 can have one or more facets that engage corresponding facets of the engagement member 624 of the driver head, such that when the engagement member 624 is disposed within the central recess 718, rotational movement of the driver 604 rotates the threaded rod 702. In some examples, the central recess 718 can be threaded and the engagement member 624 can have a correspondingly threaded shape such that the two components can be coupled together. In such examples, the central recess 718 and/or the engagement member 624 can have a circular shape in cross-section. The circumferential recess 720 of the head portion 712 can have a substantially tube-like shape with an angled/chamfered proximal surface 724 (FIG. 33), which can, for example, facilitate mating with the delivery apparatus as described below.

Rotation of the threaded rod 702 relative to the frame 700 (e.g., using an actuator assembly 600 of the delivery apparatus) results in relative axial movement between the inflow end and outflow end of the frame 700, which in turn results in radial expansion or compression of the frame. For example, moving the inflow and outflow ends toward one another (via rotation of the rod 702) causes the frame 700 to foreshorten axially and expand radially. Conversely, moving the inflow and outflow ends away from one another (via rotation of the rod 702) causes the frame 700 to elongate axially and compress radially.

Referring to FIGS. 33-35, each actuation assembly 600 can comprise a first actuation member configured as a support tube or outer sleeve 602 and a second actuation member configured as a driver 604. As shown, the driver 604 can extend through the outer sleeve/support tube 602. The distal end portions of the outer sleeve 602 and driver 604 can be configured to engage or abut the head portion 712 of the threaded rod 702 and/or an outflow end portion 722 of the frame 700.

The proximal portions of the outer sleeve 602 and driver 604 can be operatively coupled to the handle of a delivery apparatus (e.g., handle 104 of delivery apparatus 100). The delivery apparatus in this example can include the same or similar features described previously for delivery apparatus 100. In some examples, the proximal end portions of each driver 604 can be operatively connected to the knob 112 such that rotation of the knob 112 (clockwise or counterclockwise) causes corresponding rotation of the drivers 604. The proximal end portions of each outer sleeve 602 can be operatively connected to the knob 114 such that rotation of the knob 114 (clockwise or counterclockwise) causes corresponding axial movement of the sleeves 602 (proximally or distally) relative to the drivers 604. In some examples, the handle can include electric motors, buttons, switches, circuitry, etc. for actuating these components.

Referring to FIG. 31, the outer sleeve 602 can comprise a first or distal end portion 606 and an elongated body 612. The distal end portion 606 of the outer sleeve 602 can comprise first and second support extensions 608 defining gaps or notches 610 (FIG. 30) between them. Support extensions 608 and notches 610 can function in the same or a similar way as described for support extensions 534 and notches 535 described previously. In some examples, the distal end portion 606 of the outer sleeve 602 can comprise a different material and/or construction than the elongated body 612.

Referring to FIG. 33, the driver 604 can comprise an elongated body 614 and an engagement portion 616 disposed at a distal end portion of the elongated body 614. The engagement portion 616 can comprise a driver head 618 and a gripper member 620. As shown in the illustrated example, the driver head 618 and gripper member 620 can be coupled to the elongated body 614. In some examples, the driver head 618 and/or gripping portion 620 can be friction fit, welded, or otherwise coupled to the elongated body 614. In some examples, the driver head 618 and the elongated body 614 can be formed integrally with one another. The engagement portion 616 can be coupled to the elongated body 614 using any of the structures/methods described previously for gripper member 546 and/or driver head 544.

Referring to FIG. 33, the driver head 618 can comprise a main body 622 including an engagement member 624, and one or more extension members/projections 626 (FIG. 31). The one or more projections 626 can extend radially outwardly from the main body 622 and can be spaced apart from one another around a perimeter of the driver head 618. As seen in FIG. 31, the projections 626 can comprise an elongated rectangular prism shape, including chamfered side edges. In some examples, the projections 626 can have any of various other shapes.

The engagement member 624 can extend distally from the main body 622 and can be configured to engage the recess 718 of a respective threaded rod 702. Though in the illustrated example the engagement member 624 has a square shape in cross-section, in some examples, the engagement member 624 can have any of various non-circular shapes, for example, rectangular, triangular, ovular, star, etc. The recess 718 in the head portion 712 of the threaded rod 702 can be correspondingly shaped to receive the engagement member 624. The shape of the engagement member 624 and the corresponding recess 718 can, for example, advantageously improve torque transmission from the driver 604 to the threaded rod 702.

Referring now to FIG. 31, the gripper member 620 can comprise an annular base member 628 and one or more extension members/wings/arms 630 configured similar to a chuck or collet. The collet-like arms 630 can be disposed around the head portion 712 of the threaded rod 702 to releasably engage the actuator assembly 600 with the actuator mechanism 704 of the frame 700. The arms 630 can be movable between an expanded configuration (e.g., when the outer sleeve 602 is moved proximal relative to the arms 630) and a compressed configuration (e.g., when the outer sleeve 602 is advanced distally over the arms 630).

Referring to FIG. 33, the arms 630 can extend distally from the base member 628 and can each comprise a wider end portion 632 (also referred to as protrusion, projection, or tooth 632) disposed at a distal end portion of the arm 630 and extending radially inwardly toward a longitudinal axis of the driver 604. The teeth 632 can be configured (e.g., sized and shaped) to sit within the circumferential recess 720 of the head portion 712 when the actuator assembly 600 is coupled to the frame 700, as shown in FIG. 33. As shown, the tooth 632 can comprise an angled or ramped proximal surface 634 (FIG. 34), which can correspond to the chamfered surface 724 of the circumferential recess 720. Such a configuration can advantageously allow the arms 630 to easily slide into and/or out of engagement with the circumferential recess 720. As shown in FIG. 33, the distal end portion 636 of each arm 630 can flare radially outwardly (e.g., away from a longitudinal axis of the driver 604) such that the distal end portion 636 extends radially outwardly relative to a main body 638 of the arm 630 and such that an outer surface of the distal end portion 636 is spaced radially outward from an outer surface of the main body 638. In other words, the distal end portion 636 of each arm 630 can be radially thicker than the main body 638 of the arm 630, as seen in FIG. 33. Such a configuration can improve engagement of the teeth 632 with the circumferential recess 720 by increasing contact between the outer sleeve 602 and the distal end portion 636.

The arms 630 can be configured to bias radially outwardly (e.g., away from the longitudinal axis of the driver 604) into an expanded shape, for example, by shape setting the arms 630 using any of various known methods. In the illustrated example, the gripper member 620 comprises four arms, which advantageously spread the engagement forces more evenly around the circumference of the head portion 712. However, in some examples the gripper member 620 can comprise any number of arms 630, for example, one, two, three, four, five, six, seven, eight, etc.

As seen in FIG. 32, the distal end portion 636 of each arm 630 can have an increased circumferential width relative to a main body 638 of the arm 630. The increased width can advantageously improve engagement of the distal end portion 636 with the circumferential recess 720 of the head portion 712. The sectional shape of each arm 630 can be arcuate in a circumferential direction (i.e., each arm can be curved in cross-section taken in a plane perpendicular to the longitudinal axis of the arm), which can, for example, help the arm 630 resist bending in the circumferential direction. The narrower main body 638 of the arms 630 reduces the curved width such that the arms 630 can, for example, more easily bend radially (e.g., when the arms are pulled toward or away from engagement with the head portion 712).

As seen in FIG. 31, the projections 626 of the driver head 618 can extend between the arms 630 such that when the actuator assembly 600 is engaged with the frame 700, the arms 630 sit within recesses defined between the projections 626. The projections 626 can, among other things, retain the arms in the engaged configuration and/or prevent lateral displacement of the arms 630, thereby improving the stability of the arms 630 without needing to weld or otherwise affix the arms around the driver head 618. This can, for example, advantageously preserve engagement stability without the need to weld such small components.

In some examples, the actuator assembly 600 can be coupled to a respective actuator mechanism 704 of a prosthetic valve as follows. The engagement portion 616 of the driver 604 can be advanced over the head portion 712 of a threaded rod 702. The arms 630 can deflect radially outward as they are advanced over the head portion 712 until the teeth 632 are disposed within the circumferential recess 720 and the engagement member 624 of the driver 604 is disposed within the central recess 718. As the outer sleeve 602 is advanced (e.g., distally) over the driver 604, the distal end portions 636 of the arms 630 are radially compressed, thereby retaining the teeth 632 within the circumferential recess 720 and coupling the actuator assembly 600 to the threaded rod 702 (e.g., as shown in FIG. 33). The outer sleeve 602 can continue to be advanced until the support extensions 608 engage the radially inner and outer surfaces of the frame 700, as shown in FIG. 30. So coupled, the driver 604 can be rotated (e.g., using the handle of the delivery apparatus 100) to cause corresponding rotation of the threaded rod 702. The engagement between the actuator assembly 600 and the threaded rod 702 can advantageously improve torque transfer between the driver 604 and the threaded rod 702.

So coupled, the delivery apparatus and the prosthetic valve can be advanced over a guidewire through the vasculature of a patient to a selected implantation site (e.g., the native aortic annulus). For example, when implanting the prosthetic valve within the native aortic valve, the delivery apparatus and the prosthetic valve can be inserted into and through a femoral artery, through the aorta, and to the native aortic valve. The prosthetic valve can then be deployed at the implantation site (e.g., within the native aortic valve) and can be expanded and locked in the expanded configuration using the actuator mechanisms 704.

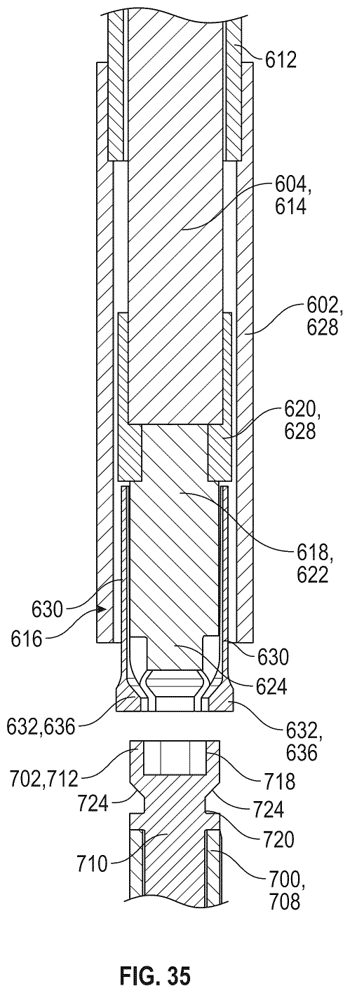

Referring to FIGS. 30-32 (FIGS. 33-35 show a cross-sectional view of the same process), once final positioning and expansion of the prosthetic valve is achieved, the actuator assemblies 600 can be released from the prosthetic valve in the following exemplary manner. The outer sleeve 602 can be retracted to uncover the connection between the driver 604 and the threaded rod 702, as shown in FIGS. 31 and 34. With the outer sleeve proximal relative to the distal end portions 636 of the arms 630, the arms 630 can move radially outwardly (e.g., due to being shape-set to a radially expanded/disengaged state and/or by contact with the head portion 712 of the threaded rod 702). The angled surfaces 724 of the head portion 712 can, for example, help facilitate radial expansion of the arms 630 as the arms 630 move proximally from the circumferential recess 720. The driver 604 can then be retracted as shown in FIGS. 32 and 35, removing the engagement member 624 from within the recess 718 of the rod 702, thereby decoupling the driver 604 from the rod 702. At this stage, the delivery apparatus (including the actuator assemblies 600) can be retracted relative to the prosthetic valve and removed from the patient's body.

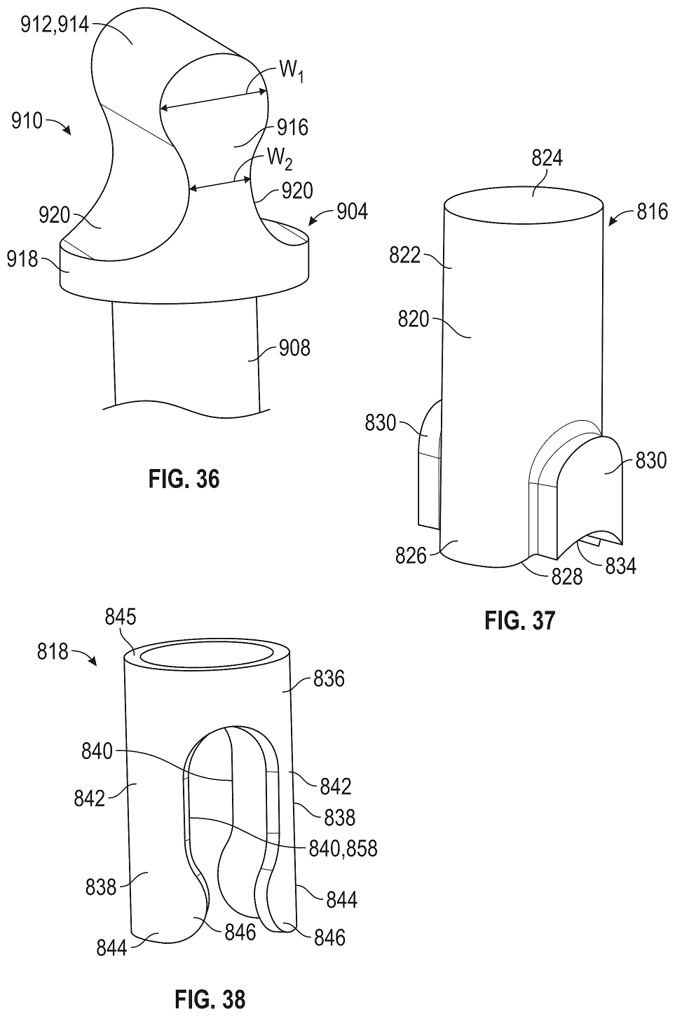

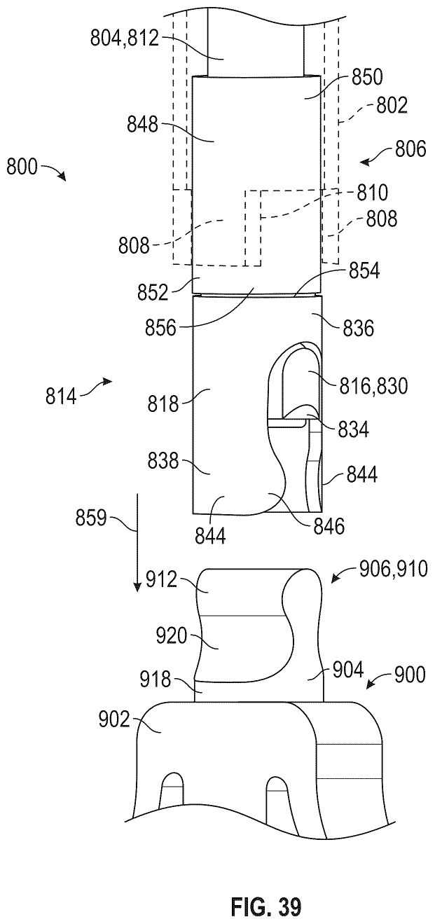

FIGS. 36-41 illustrate another example of an actuator assembly 800 (FIG. 39) of a delivery apparatus that can be used to radially expand and/or compress a prosthetic valve. Actuator assembly 800 can be similar to actuator assemblies 500 and 600 described previously, except that the one or more arms/extension members/wings 838 of the gripper member 818 can have a uniform thickness, which advantageously makes them easier to manufacture without the need for rod turning procedures. The gripper member 818 can also be configured to advantageously be coupled to the driver 804 without the need for welding, as described in more detail below.

FIGS. 39-41 illustrate an actuator assembly 800 being used on a portion of an exemplary frame 902 of a prosthetic valve 900, however, the actuator assembly 800 can be used on any of the prosthetic valves described herein and/or on any other prosthetic valve having a similar expansion mechanism. Though only a portion of the frame 902 is shown, it should be appreciated that the frame 902 is an annular structure that can support a valvular structure and can comprise inner and/or outer skirts to form a prosthetic heart valve 900 similar to prosthetic heart valve 400.

The actuator assembly 800 can be a component of a delivery apparatus (such as delivery apparatus 100, described previously), and can be configured to releasably couple and actuate a respective actuator/screw/actuation bolt/threaded rod of prosthetic valve 900, such as threaded rod 904 of actuator mechanism 906, partially shown in FIGS. 39-41. Actuator mechanism 906 can be the same as or similar to actuator mechanisms 206, 406, and/or 704 described previously. The rod 904 can be used in lieu of or in addition to rods 234, 426, and/or 702 of prosthetic valves 200, 400, and 700 described herein. The delivery apparatus can include a plurality of actuator assemblies 800, one for each rod 904 of the prosthetic valve 900. The delivery apparatus can include any number of actuator assemblies (e.g., 1-15 actuator assemblies). The number of actuation assemblies of the delivery apparatus can, for example, be determined by the number of threaded rods of the prosthetic valve for which the delivery apparatus is being used to implant.

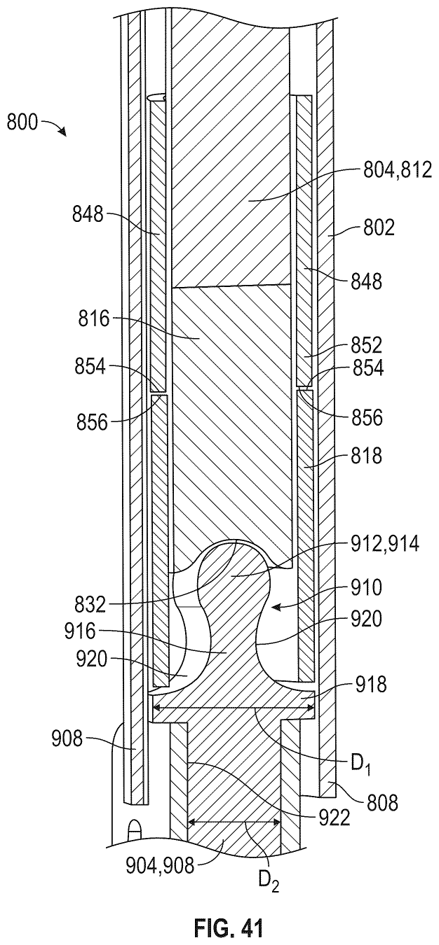

Referring to FIG. 36, the rod 904 can comprise an elongated body 908 (partially shown) and a head portion 910 configured to be releasably coupled to a respective actuator assembly 800. The elongated body 908 can comprise a threaded portion (not shown). The head portion 910 can have an overall 22-shape comprising a projection 912 including a projection head 914 and a projection neck 916, and a flared base portion 918. The projection head 914 can have a width W₁ greater than a width W₂ of the projection neck 916. The projection 912 and the flared base portion 918 can together define first and second curved recesses 920 on opposing sides of the head portion 910. As shown in FIG. 41, the flared base portion 918 can have a diameter D₁ greater than a diameter D₂ of the opening 922 in the frame 902 such that the head portion 910 is prevented from moving into the frame 902 and such that the head portion 910 abuts the outflow edge of the frame as shown in, e.g., FIG. 41. The head portion 910 of the rod 904 can be used to apply a distally-directed force to the frame 902, for example, during radial expansion of the frame 902.

Rotation of the threaded rod 904 relative to the frame 902 (e.g., using an actuator assembly 800 of the delivery apparatus) results in relative axial movement between the inflow end and outflow end of the frame 902, which in turn results in radial expansion or compression of the frame. For example, moving the inflow and outflow ends toward one another (via rotation of the rod 904 in a first direction) causes the frame 902 to foreshorten axially and expand radially. Conversely, moving the inflow and outflow ends away from one another (via rotation of the rod 904 in a second direction) causes the frame 902 to elongate axially and compress radially.

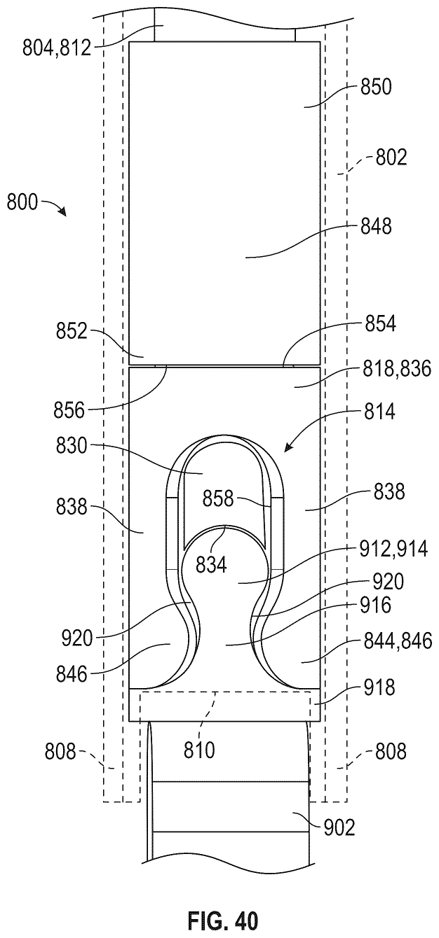

Referring to FIGS. 39-41, each actuation assembly 800 can comprise a first actuation member configured as a support tube or outer sleeve 802 (shown in dashed lines in FIGS. 39-40) and a second actuation member configured as a driver 804. The driver 804 can extend through the outer sleeve 802 as shown. The distal end portions of the outer sleeve 802 and driver 804 can be configured to engage or abut the head portion 910 of the rod 904 and/or a portion of the frame 902.

The proximal portions of the outer sleeve 802 and driver 804 can be operatively coupled to the handle of a delivery apparatus (e.g., handle 104 of delivery apparatus 100). The delivery apparatus in this example can include the same or similar features described previously for delivery apparatus 100. In some examples, the proximal end portions of each driver 804 can be operatively connected to the knob 112 such that rotation of the knob 112 (clockwise or counterclockwise) causes corresponding rotation of the drivers 804. The proximal end portions of each outer sleeve 802 can be operatively connected to the knob 114 such that rotation of the knob 114 (clockwise or counterclockwise) causes corresponding axial movement of the sleeves 802 (proximally or distally) relative to the drivers 804. In some examples, the handle can include electric motors, buttons, switches, circuitry, etc. for actuating these components.

Referring to FIG. 39, the distal end portion 806 of the outer sleeve 802 can comprise first and second support extensions 808 defining gaps or notches 810 between them. Support extensions 808 and notches 810 can function in the same or a similar way as described for support extensions 534/608 and notches 535/610 described previously.

The driver 804 can comprise an elongated body 812 (FIG. 41) and an engagement portion 814 disposed at a distal end portion thereof. The engagement portion 814 can comprise a driver head 816 and a gripper member 818.

Referring to FIG. 37, the driver head 816 can comprise a main body 820 including a first end portion 822 having a first end surface 824 and a second end portion 826 having a second end surface 828, and first and second shoulders 830 extending radially outwardly from the second end portion 826. In some examples, as shown, the main body 820 can have a substantially cylindrical shape.

As shown in FIG. 41, the second end surface 828 can comprise an engagement recess 832 extending into the main body 820 and configured (e.g., sized and shaped) to engage the projection head 914 of the rod 904. The engagement recess 832 can have a substantially semi-circular shape in cross section. Referring to FIGS. 37 and 39-40, the shoulders 830 can comprise cutouts 834, such as semi-circular shaped cutouts, configured (e.g., sized and shaped) to engage the projection head 914. In some examples, the engagement recess 832 and cutouts 834 can have any of various shaped in cross-section such that they are configured to receive the projection 912 of the rod 904. The shapes of the engagement recess 832, cutouts 834, and the corresponding projection 912 can, for example, advantageously improve torque transmission from the driver 804 to the rod 904.

Referring now to FIG. 38, the gripper member 818 can comprise an annular base member 836 and one or more extension members/wings/arms 838. In the illustrated example, the gripper member 818 has first and second diametrically opposed arms 838 defining first and second diametrically opposed recesses 840 between them. The recesses 840 can have an Ω-shape such that they substantially correspond with the Ω-shape of the head portion 910 of the rod 904. In some examples, the gripper member 818 can have any number of arms and any number of recesses. In some examples, the arms 838 can be movable between an expanded configuration (e.g., when the outer sleeve 802 is moved to a proximal position relative to the arms 838) and a compressed configuration (e.g., when the outer sleeve 802 is advanced distally and positioned over the arms 838).

As shown, the arms 838 can extend distally from the base member 836 and can comprise a proximal neck portion 842 having a first circumferential width, and a distal end portion 844 having a second, wider circumferential width. The distal end portion 844 can comprise circumferentially-extending curved protrusions 846 configured to be disposed within the curved recesses 920 of the rod head 910 such that the gripper member 818 engages the rod 904, as shown in FIG. 40. The increased width of the distal end portions 844 including the curved protrusions 846, can advantageously improve engagement of the distal end portion 844 with the curved recesses 920 of the rod head 910. The sectional shape of each arm 838 can be arcuate in a circumferential direction (i.e., each arm can be curved in cross-section taken in a plane perpendicular to the longitudinal axis of the arm), which can, for example, help the arm 838 resist bending in the circumferential direction. The narrower neck portion 842 of the arms 838 reduces the curved width such that the arms 838 can, for example, more easily bend radially (e.g., when the arms are pulled toward or away from engagement with the head portion 910).

As can be seen for example in FIG. 41, unlike the arms 630 of gripper member 620, arms 838 have a uniform thickness from the base member 836 to the distal end portion 844. Such a configuration advantageously allows for easier manufacture of the gripper member 818, for example using laser-cutting and shape-setting (e.g., heat-setting) of the arms, without the need for rod turning procedures. In some examples, the gripper member 818 can comprise Nitinol. The arms 838 can be configured to bias radially outwardly (e.g., away from a longitudinal axis of the driver 804) into an expanded shape, for example, by shape-setting (e.g., heat-setting) the arms 838 using any of various known methods. In the illustrated example, the gripper member 818 comprises two arms 838, however, in some examples the gripper member can comprise any number of arms.

The driver head 816 and gripper member 818 can each be coupled to the elongated body 812 of the driver 804. For example, as shown in FIGS. 39-41, the driver 804 can include a coupling member 848 such as a cylindrical tube, that is coupled (e.g., welded) at a first end portion 850 to the elongated body 812 and at a second end portion 852 to the driver head 816. As can be seen in, for example, FIG. 41, the gripper member 818 can be disposed over the driver head 816 at a location distal to the coupling member 848 such that a proximal edge 854 of the annular base member 836 abuts a distal edge 856 of the coupling member 848. As shown in FIG. 40, this positions the shoulders 830 of the driver head 816 within respective proximal portions 858 of the respective recesses 840. The gripper member 818 is thereby restrained from axial and rotational movement relative to the driver head 816 and elongated body 812 by the engagement of the shoulders 830 and the distal edge portion 856 of the coupling member 848. The gripper member 818 is thus advantageously coupled to the elongated body 812 without the need for welding, which simplifies the manufacturing process.

In some examples, the driver head 816 and/or gripper member 818 can be friction fit, welded, or otherwise coupled to the elongated body 812. In some examples, the driver head 816 and the elongated body 812 can be formed integrally with one another. In some examples, the engagement portion 814 can be coupled to the elongated body 812 using any of the structures/methods described previously for gripper members 546/620 and/or driver heads 544/618.

In some examples, the actuator assembly 800 can be coupled to a respective actuator mechanism 906 of a prosthetic valve as follows. Referring to FIG. 39, the engagement portion 814 of the driver 804 can be advanced over the head portion 910 of the rod 904 in the direction shown by arrow 859. The arms 838 can deflect radially outward as they are advanced over the head portion 910 until the curved protrusions 846 are disposed within the curved recesses 920 of the rod head 910 and the engagement recess 832 of the driver head 816 engages the projection head 914 of the rod. As the outer sleeve 802 is advanced (e.g., distally) over the engagement portion 814, the distal end portions 844 of the arms 838 are radially compressed, thereby retaining the curved protrusions 846 within the curved recesses 920 and coupling the actuator assembly 800 to the rod 904 (e.g., as shown in FIGS. 40-41).

The outer sleeve 802 can continue to be advanced until the support extensions 808 engage the radially inner and outer surfaces of the frame 902, as shown, for example, in FIG. 40. So coupled, the driver 804 can be rotated (e.g., using the handle of the delivery apparatus 100) to cause corresponding rotation of the rod 904. The engagement between the actuator assembly 800 and the rod 904 can advantageously improve torque transfer between the driver 804 and the threaded rod 904.

So coupled, the delivery apparatus and the prosthetic valve can be advanced over a guidewire through the vasculature of a patient to a selected implantation site (e.g., the native aortic annulus). For example, when implanting the prosthetic valve within the native aortic valve, the delivery apparatus and the prosthetic valve can be inserted into and through a femoral artery, through the aorta, and to the native aortic valve. The prosthetic valve can then be deployed at the implantation site (e.g., within the native aortic valve) and can be expanded and locked in the expanded configuration using the actuator mechanisms 704.

Once final positioning and expansion of the prosthetic valve is achieved, the actuator assemblies 800 can be released from the prosthetic valve in the following exemplary manner. The outer sleeve 802 can be retracted to uncover the connection between the driver 804 and the rod 904. With the outer sleeve proximal relative to the distal end portions 844 of the arms 838, the arms 838 can move radially outwardly (e.g., due to being shape-set to a radially expanded/disengaged state). The driver 804 can then be retracted, removing the engagement recess 832 of the driver head 816 from the projection head 914, thereby decoupling the driver 804 from the rod 904. At this stage, the delivery apparatus (including the actuator assemblies 800) can be retracted relative to the prosthetic valve and removed from the patient's body.

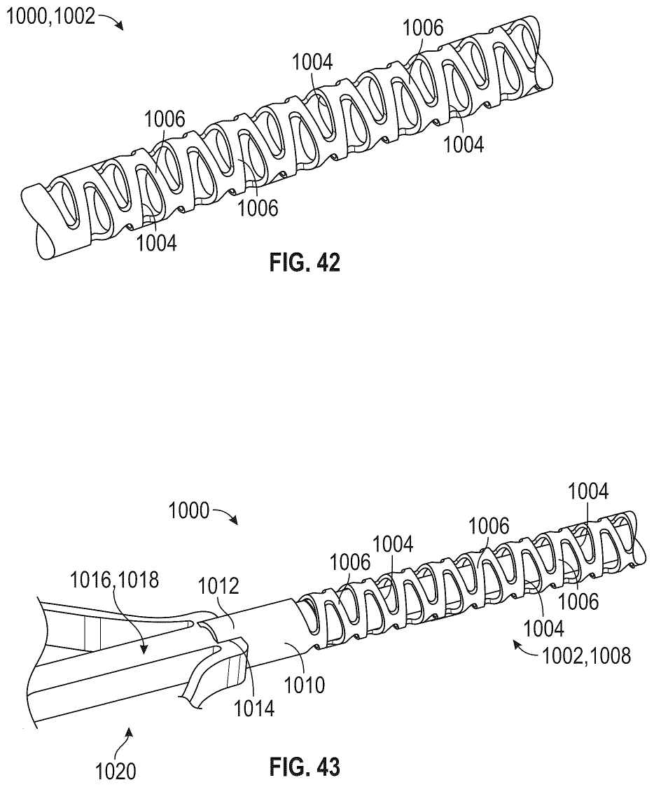

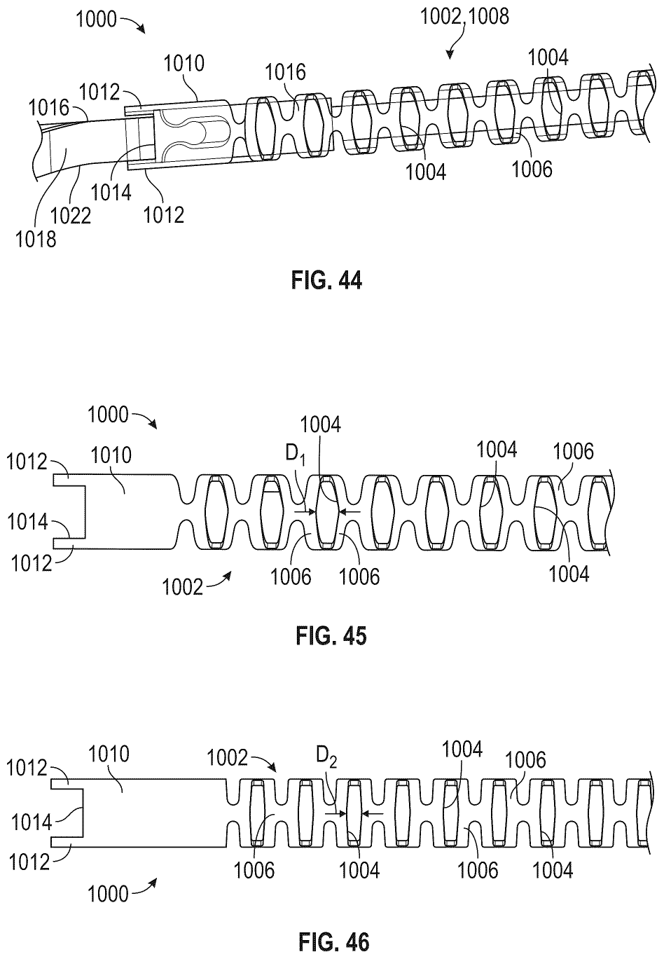

In some cases, the outer sleeve (such as outer sleeve 302, 524, 602, or 802 described previously) can inadvertently slip or bend during delivery or use, resulting in premature release of the arms and inadvertent disengagement of the actuation assembly from the frame. Referring to FIGS. 42-46, in some examples, in lieu of or in addition to outer sleeves 302, 524, 602, and 802 described previously, any of the actuation assemblies described herein can comprise an outer sleeve 1000, at least a portion of which is configured as a hypotube 1002. FIG. 44 shows the outer sleeve 1000 transparently for purposes of illustration. The hypotube 1002 can be expandable and compressible between a first length and a second length. During use, the outer sleeve 1000 can initially be disposed around a driver (e.g., any of the drivers described herein) in a compressed configuration such that even if the outer sleeve 1000 and driver bend during delivery or use, the outer sleeve 1000 can advantageously bias into an expanded configuration wherein the arms of the driver remain covered and thus restrained.

As shown in FIG. 42, the hypotube 1002 can comprise a plurality of compressible and expandable diamond-shaped openings or slots 1004 that extend at least partially around the circumference of the outer sleeve 1000. The slots 1004 can be axially separated from one another by circumferentially-extending members or ribs 1006. As mentioned, the hypotube 1002 can be movable between an expanded configuration having a first overall length, wherein the ribs are separated from each other by a first distance D₁ (FIG. 45), and a compressed configuration wherein the ribs are separated from each other by a second distance D₂ (FIG. 46) shorter than the first distance. Though the difference between D₁ and D₂ can be very slight (e.g., on the order of microns), the combined expansion of one or more slots 1004 can result in an overall elongation of the hypotube portion 1002 (e.g., up to on the order of several centimeters). The hypotube 1002 can be configured to bias into the expanded configuration. In some examples, the hypotube 1002 can be a laser cut tube.

The diamond-shaped slots 1004 can be configured (e.g., sized and shaped) to provide the desired elongation and resulting force. For example, the slots 1004 can be configured to provide a low force yet a greater elongation. The axial pre-loading of the hypotube portion prevents or mitigates unwanted movement of the outer sleeve (e.g., caused by bending during navigation through the patient's anatomy) by allowing the hypotube to expand (e.g., in a compression-spring like fashion) when there is space available to do so. In some cases unwanted movement can undesirably cause the actuator assembly to decouple from the prosthetic valve, and thus it is important to mitigate such movement.

In some examples, the outer sleeve 1000 can further function to visually indicate to a physician that the actuator assembly has successfully released from the prosthetic valve. For example, this can be done by releasing a mechanical latch at a proximal end of the loaded outer sleeve. Once the latch is released, the proximal end portion of the outer sleeve can expand in a proximal direction (e.g., toward the physician) and not only release the outer sleeve from the prosthetic valve but also move the entire outer sleeve in a proximal direction, thus providing a visual gap between a distal end of the actuator assembly and the prosthetic heart valve that would be visible on the x-ray screen.

As shown, for example, in FIG. 43, the outer sleeve 1000 can comprise a first, elongated portion 1008 that comprises the hypotube 1002, and a non-hypotube second portion 1010 at a distal end thereof. The second portion 1010 can comprise first and second support extensions 1012 defining gaps or notches 1014 between them. Support extensions 1012 and notches 1014 can function in the same or a similar way as described for support extensions 608/534/808 and notches 610/535/810 described previously. For example, as shown in FIG. 43, a first support extension 1012 can extend partially over a radially inner surface 1016 of a frame 1018 of a prosthetic valve 1020 and the second support extension 1012 can extend partially over a radially outer surface 1022 of the frame 1018 to inhibit rotation of the frame 1018 relative to the actuator assemblies. In some examples, the distal end portion 1010 of the outer sleeve 1000 can comprise a different material and/or construction than the first portion.

During delivery and/or use, the outer sleeve 1000 comprising hypotube 1002 can be axially pre-loaded such that it is disposed over a driver (such as drivers 304, 526, 604, or 804 described herein) in the compressed configuration. If the outer sleeve bends during delivery or use, the hypotube portion 1002 advantageously expands, allowing the distal end portion 1010 to remain in position over the arms, thus preventing the arms from inadvertent expansion.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An assembly, comprising:
a prosthetic heart valve comprising:
   a radially expandable and compressible frame having an inflow end portion, an outflow end portion, and a plurality of actuator mechanisms, each actuator mechanism comprising:
      a first frame member having a first inner bore and a second frame member having a second inner bore, the first and second frame members being spaced apart axially from one another, and
      an actuator having an external threaded surface and extending through the first and second inner bores; and
a delivery apparatus comprising:
   a handle,
   one or more actuator assemblies extending from the handle, each actuator assembly comprising:
   a first actuation member having a distal end portion releasably coupled to the outflow end portion of the frame, the distal end portion comprising first and second support extensions; and
   a second actuation member extending through the first actuation member and comprising a distal end portion having an engagement portion releasably coupled to the rod;
wherein rotation of the second actuation member in a first direction causes corresponding rotation of the rod such that the first and second members move axially toward one another to expand the prosthetic valve; and
wherein the first and second support extensions inhibit rotation of the frame relative to the one or more actuator assemblies during expansion of the prosthetic valve.

Example 2. The assembly of any example herein, particularly example 1, wherein the first support extension extends partially over a radially inner surface of the frame and the second support extension extends partially over a radially outer surface of the frame.

Example 3. The assembly of any of any example herein, particularly any one of examples 1-2, wherein a proximal end portion of the actuator comprises first and second protrusions defining a slot between them.

Example 4. The assembly of any example herein, particularly example 3, wherein the engagement portion comprises a central protrusion extending into the slot.

Example 5. The assembly of any example herein, particularly example 4, wherein the central protrusion is sized such that it does not contact an inner surface of the first actuation member.

Example 6. The assembly of any example herein, particularly any one of examples 1-5, wherein the proximal end portion of the actuator comprises one or more shoulders extending radially from a surface of the actuator.

Example 7. The assembly of any example herein, particularly example 6, wherein the engagement portion comprises one or more flexible elongated elements releasably coupled to the shoulders.

Example 8. The assembly of any example herein, particularly example 7, wherein each elongated element comprises a protrusion extending radially inwardly toward a longitudinal axis of the second actuation member.

Example 9. The assembly of any example herein, particularly example 7, wherein the one or more elongated elements bias radially outwardly from a longitudinal axis of the second actuation member.

Example 10. The assembly of any example herein, particularly any one of examples 1-9, wherein rotation of the second actuation member in a second direction causes corresponding rotation of the actuator such that the first and second frame members move axially away from one another to radially compress the prosthetic valve.

Example 11. The assembly of any example herein, particularly any one of examples 1-10, each actuator comprising a stopper disposed axially between the first and second frame members, the stopper configured to selectively abut an inflow end portion of the second frame member to prevent over crimping of the frame.

Example 12. The assembly of any example herein, particularly any one of examples 1-11, each first frame member comprising a nut disposed at an outflow end portion of the first frame member, the nut comprising an inner threaded bore configured to engage the external threaded surface of the actuator.

Example 13. The assembly of any example herein, particularly example 12, wherein the nut is visible through a window extending through a wall of the first member.

Example 14. The assembly of any example herein, particularly any one of examples 1-13, the frame comprising a plurality of circumferentially disposed hexagonal cells.

Example 15. The assembly of any example herein, particularly example 14, wherein each hexagonal cell comprises a diamond cell disposed within an outer perimeter of the hexagonal cell.

Example 16. The assembly of any example herein, particularly example 15, wherein each hexagonal cell and diamond cell comprises an inflow apex and an outflow apex, and wherein each actuator extends through the inflow and outflow apices.

Example 17. The assembly of any example herein, particularly any one of examples 1-16, wherein the frame further comprises one or more axially extending support posts.

Example 18. The assembly of any example herein, particularly any one of examples 1-17, wherein the plurality of actuator mechanisms are each coupled to one or more support posts by a plurality of struts.

Example 19. The assembly of any example herein, particularly example 18, wherein the struts are curved.

Example 20. The assembly of any example herein, particularly any one of examples 18-19, wherein each strut has a recurve shape including a first portion and a second portion separated by an inflection point.

Example 21. The assembly of any example herein, particularly example 20, wherein the first portion is an upwardly curved portion, and the second portion is a downwardly curved portion.

Example 22. The assembly of any example herein, particularly any one of examples 18-21, wherein each strut comprises a first end portion and a second end portion and wherein the first and second end portions terminate asymptotically against at least one of a respective support post and actuator mechanism.

Example 23. The assembly of any example herein, particularly any one of examples 1-22, wherein each actuator is configured as at least one of a right-handed rod and a left-handed rod.

Example 24. The assembly of any example herein, particularly example 23, wherein the prosthetic heart valve comprises six actuator mechanisms and wherein the actuators of the actuator mechanisms alternate between right-handed rods and left-handed rods about the circumference of the frame.

Example 25. An assembly, comprising:
a prosthetic heart valve comprising:
   a radially expandable and compressible frame having an inflow end portion and an outflow end portion, the frame comprising a plurality of actuation mechanisms, each actuation mechanism comprising:
   a first frame member having a first inner bore and a second frame member having a second inner bore, the first and second frame members being spaced apart axially from one another, and
   a rod having an external threaded surface and extending through the first and second inner bores, a proximal end portion of the rod comprising first and second protrusions defining a slot between them, and first and second shoulders; and
a delivery apparatus comprising:
   a handle,
   one or more actuator assemblies extending from the handle, each actuator assembly comprising:
   a first actuation member having a distal end portion abutting the outflow end portion of the frame, and
   a second actuation member extending through the first actuation member, the second actuation member comprising a distal end portion having a central protrusion extending into the slot and first and second flexible elongated elements releasably coupled to the shoulders; and
wherein rotation of the second actuation member in a first direction causes corresponding rotation of the rod such that the first and second frame members move axially toward one another to expand the prosthetic valve.

Example 26. The assembly of any example herein, particularly example 25, wherein the first actuation member further comprises first and second support extensions configured to inhibit rotation of the frame relative to the first actuation member during expansion of the frame.

Example 27. The assembly of any example herein, particularly example 26, wherein the first support extension extends partially over a radially inner surface of the frame and the second support extension extends partially over a radially outer surface of the frame.

Example 28. The assembly of any example herein, particularly any one of examples 25-27, wherein the central protrusion is sized such that it does not contact an inner surface of the first actuation member.

Example 29. The assembly of any example herein, particularly any one of examples 25-28, wherein each elongated element comprises a radially inwardly extending protrusion.

Example 30. The assembly of any example herein, particularly example 29, wherein the one or more elongated elements bias radially outwardly from a longitudinal axis of the second actuation member.

Example 31. The assembly of any example herein, particularly any one of examples 25-30, wherein rotation of the second actuation member in a second direction causes corresponding rotation of the rod such that the first and second frame members move axially away from one another to radially compress the prosthetic valve.

Example 32. The assembly of any example herein, particularly any one of examples 25-31, each rod comprising a stopper disposed between the first and second frame members, the stopper configured to selectively abut an inflow end portion of the second frame member to prevent over crimping of the frame.

Example 33. The assembly of any example herein, particularly any one of examples 25-32, each first frame member comprising a nut disposed at an outflow end portion of the first member, the nut comprising an inner threaded bore configured to engage the threaded rod.

Example 34. The assembly of any example herein, particularly example 33, wherein the nut is visible through a window extending through a wall of the first frame member.

Example 35. The assembly of any example herein, particularly any one of examples 25-34, further comprising a plurality of posts;

wherein one or more of the posts are configured as the actuation mechanisms; and wherein the plurality of posts are coupled to one another by a plurality of struts.

Example 36. The assembly of any example herein, particularly example 35, wherein the struts are curved.

Example 37. The assembly of any example herein, particularly any one of examples 25-26, wherein each strut has a recurve shape including a first portion and a second portion separated by an inflection point.

Example 38. The assembly of any example herein, particularly example 37, wherein the first portion is an upwardly curved portion and the second portion is a downwardly curved portion.

Example 39. The assembly of any example herein, particularly any one of examples 25-38, wherein each strut comprises a first end portion and a second end portion and wherein the first and second end portions terminate asymptotically against respective posts.

Example 40. A delivery apparatus, comprising:
a handle,
one or more actuator assemblies extending from the handle, each actuator assembly comprising:
a first actuation member having a distal end portion configured to abut an outflow end portion of a prosthetic heart valve, the distal end portion comprising first and second support extensions; and
a second actuation member extending through the first actuation member and having a distal end portion configured to releasably couple an actuator of the prosthetic heart valve;
wherein rotation of the second actuation member in a first direction radially expands the prosthetic valve and rotation of the second actuation member in a second direction radially compresses the prosthetic valve; and
wherein the first support extension is configured to extend partially over a radially inner surface of the prosthetic valve and the second support extension is configured to extend partially over a radially outer surface of the prosthetic valve to inhibit rotation of the frame of the prosthetic valve relative to the actuator assemblies.

Example 41. The delivery apparatus of any example herein, particularly example 40, wherein the second actuation member comprises a central protrusion.

Example 42. The delivery apparatus of any example herein, particularly example 41, wherein the central protrusion is sized such that it does not contact an inner surface of the first actuation member.

Example 43. The delivery apparatus of any example herein, particularly any one of examples 40-42, wherein the distal end portion of the second actuation member comprises one or more flexible elongated elements.

Example 44. The delivery apparatus of any example herein, particularly example 43, wherein each elongated element comprises a protrusion extending radially inwardly toward a longitudinal axis of the second actuation member.

Example 45. The delivery apparatus of any example herein, particularly example 44, wherein the one or more elongated elements bias radially outwardly from a longitudinal axis of the second actuation member.

Example 46. An implantable prosthetic device, comprising:
a radially expandable and compressible frame having an inflow end portion and an outflow end portion, the frame comprising:
   a plurality of posts, one or more of which are configured as actuation mechanisms comprising:
      a first frame member having a first inner bore,
      a second frame member having a second inner bore, the first and second frame members being spaced apart axially from one another, and
      an actuator having an external threaded surface and extending through the first and second inner bores, a proximal end portion of the actuator comprising first and second protrusions defining a slot between them, and first and second shoulders extending radially from an outer surface of the actuator; and
a plurality of struts coupling adjacent posts to one another; and
wherein rotation of the actuator in a first direction results in axial movement of the first and second frame members toward one another to radially expand the prosthetic device.

Example 47. The prosthetic device of any example herein, particularly example 46, further comprising a valvular structure comprising a plurality of leaflets disposed within the frame.

Example 48. The prosthetic device of any example herein, particularly any one of examples 46-47, wherein rotation of the actuator in a second direction results in axial movement of the first and second members away from one another to radially compress the prosthetic device.

Example 49. The prosthetic device of any example herein, particularly any one of examples 46-48, each actuator comprising a stopper disposed axially between the first and second frame members, the stopper configured to selectively abut an inflow end portion of the second frame member to prevent over crimping of the frame.

Example 50. The prosthetic device of any example herein, particularly any one of examples 46-49, each first frame member comprising a nut disposed at an outflow end portion of the first frame member, the nut comprising an inner threaded bore configured to engage the external threaded surface of the rod.

Example 51. The any example herein, particularly example 50, wherein the nut is visible through a window extending through a wall of the first frame member.

Example 52. The prosthetic device of any example herein, particularly any one of examples 46-51, the struts and posts defining a plurality of circumferentially disposed hexagonal cells.

Example 53. The prosthetic device of any example herein, particularly example 52, wherein each hexagonal cell comprises a diamond cell disposed within an outer perimeter of the hexagonal cell.

Example 54. The prosthetic device of claim 53, wherein each hexagonal cell and diamond cell comprises an inflow apex and an outflow apex, and wherein each actuation mechanism extends through the inflow and outflow apices.

Example 55. The prosthetic device of any example herein, particularly any one of examples 46-54, wherein the struts are curved.

Example 56. The prosthetic device of any example herein, particularly any one of examples 46-55, wherein each strut has a recurve shape including a first portion and a second portion separated by an inflection point.

Example 57. The prosthetic device of any example herein, particularly example 56, wherein the first portion is an upwardly curved portion and the second portion is a downwardly curved portion.

Example 58. The prosthetic device of any example herein, particularly any one of examples 46-57, wherein each strut comprises a first end portion and a second end portion and wherein the first and second end portions terminate asymptotically against respective posts.

Example 59. A method, comprising:
inserting a distal end of a delivery apparatus into the vasculature of a patient, the delivery apparatus releasably coupled to a prosthetic valve via a plurality of actuator assemblies, the prosthetic heart valve including a frame comprising a plurality of actuation mechanisms each comprising a first frame member, a second frame member axially spaced from the first frame member, and an actuator extending through the first and second frame members, each actuator assembly comprising a first actuation member engaging an outflow end of the prosthetic valve and a second actuation member extending through the first actuation member and engaging an outflow end of the actuator, the first actuation member comprising a first support extension extending partially over a radially inner surface of the frame and a second support extension extending partially over a radially over surface of the frame;
advancing the prosthetic valve to a selected implantation site; and
rotating the second actuation member to cause corresponding rotation of the actuator resulting in axial movement of the first and second frame members toward one another to radially expand the prosthetic valve, the first and second support extensions inhibiting rotation of the frame relative to the first actuation member during expansion.

Example 60. The method of any example herein, particularly example 59, further comprising:
rotating the second actuation member in a second direction to cause corresponding rotation of the actuator resulting in axial movement of the first and second frame members away from one another to radially compress the prosthetic valve, the first and second support extensions inhibiting rotation of the frame relative to the first actuation member during compression of the frame.

Example 61. The method of any example herein, particularly any one of examples 59-60, wherein the actuator comprises a stopper disposed between the first and second frame members, and wherein during radial compression of the frame the stopper selectively abuts an inflow end of the second frame member.

Example 62. An assembly, comprising:
a prosthetic heart valve comprising:
   a radially expandable and compressible frame having an inflow end portion and an outflow end portion, and
   one or more actuators having an external threaded surface and configured to radially expand the frame upon rotation of the one or more actuators; and a delivery apparatus comprising:
   a handle,
   one or more actuator assemblies extending from the handle, each actuator assembly comprising:
      a first actuation member having a distal end portion releasably coupled to the outflow end portion of the frame, the distal end portion comprising first and second support extensions; and
      a second actuation member extending through the first actuation member and comprising a distal end portion having an engagement portion, the engagement portion comprising:
         a driver head having an engagement member that extends into a corresponding recess in a corresponding actuator, and
         a gripper member comprising one or more arms releasably coupled to one or more projections extending from the actuator;
wherein rotation of the second actuation member in a first direction causes corresponding rotation of the actuator to expand the prosthetic valve; and
wherein the first and second support extensions inhibit rotation of the frame relative to the one or more actuator assemblies during expansion of the prosthetic valve.

Example 63. The assembly of any example herein, particularly example 62, wherein the first support extension extends partially over a radially inner surface of the frame and the second support extension extends partially over a radially outer surface of the frame.

Example 64. The assembly of any example herein, particularly any one of examples 62-63, wherein the engagement portion comprises a square shape with chamfered corners in cross-section.

Example 65. The assembly of any example herein, particularly example 64, wherein the engagement member extends distally from the driver head.

Example 66. The assembly of any example herein, particularly any one of examples 62-65, wherein the engagement portion is sized such that it does not contact an inner surface of the first actuation member.

Example 67. The assembly of any example herein, particularly any one of examples 62-66, wherein the arms are movable between an expanded position and a compressed position.

Example 68. The assembly of any example herein, particularly example 67, wherein the arms bias radially outwardly from a longitudinal axis of the second actuation member.

Example 69. The assembly of any example herein, particularly any one of examples 62-68, wherein each arm comprises an opening, and wherein a respective projection of the actuator is selectively disposed within the opening.

Example 70. The assembly of any example herein, particularly any one of examples 62-69, wherein rotation of the second actuation member in a second direction causes corresponding rotation of the actuator to radially compress the prosthetic valve.

Example 71. The assembly of any example herein, particularly any one of examples 62-70 wherein the frame comprises one or more pairs of axially spaced first and second frame members, each actuator extending through the first and second frame members of a corresponding pair.

Example 72. The assembly of any example herein, particularly example 71, wherein each actuator comprises a stopper disposed axially between the first and second frame members of a corresponding pair, the stopper configured to selectively abut an inflow end portion of the second frame member.

Example 73. The assembly of any example herein, particularly any one of examples 71-72, wherein each first frame member comprises a nut disposed at an outflow end portion of the first frame member, the nut comprising an inner threaded bore configured to engage a corresponding actuator.

Example 74. A delivery apparatus, comprising:
a handle;
one or more actuator assemblies extending from the handle, each actuator assembly comprising:
   a first actuation member having a distal end portion configured to abut an end portion of a prosthetic heart valve, the distal end portion comprising first and second support extensions,
   a second actuation member extending through the first actuation member and comprising a driver head having an engagement member configured to engage a corresponding engagement portion of an actuator of the prosthetic heart valve, and a gripper member comprising one or more arms configured to releasably couple the actuator; and
wherein rotation of the second actuation member in a first direction is configured to radially expand the prosthetic valve and rotation of the second actuation member in a second direction is configured to radially compresses the prosthetic valve.

Example 75. The delivery apparatus of any example herein, particularly example 74, wherein the first support extension is configured to extend partially over a radially inner surface of the prosthetic valve and the second support extension is configured to extend partially over a radially outer surface of the prosthetic valve to inhibit rotation of the frame of the prosthetic valve relative to the actuator assemblies.

Example 76. The delivery apparatus of any example herein, particularly any one of examples 74-75, wherein the engagement portion comprises a square shape with chamfered corners in cross-section.

Example 77. The delivery apparatus of any example herein, particularly example 76, wherein the engagement member extends distally from the driver head.

Example 78. The delivery apparatus of any example herein, particularly any one of examples 74-77, wherein the second actuation member is sized such that it does not contact an inner surface of the first actuation member.

Example 79. The delivery apparatus of any example herein, particularly, any one of examples 74-78, wherein the one or more arms comprise a plurality of arms.

Example 80. The delivery apparatus of any example herein, particularly example 79, wherein the arms are movable between an expanded position and a compressed position.

Example 81. The delivery apparatus of any example herein, particularly example 80, wherein the arms bias radially outwardly from a longitudinal axis of the second actuation member.

Example 82. The delivery apparatus of any example herein, particularly any one of examples 74-81, wherein each arm comprises an opening configured to selectively couple a respective projection of the actuator.

Example 83. The delivery apparatus of any example herein, particularly any one of examples 74-82, wherein rotation of the second actuation member in a second direction is configured to cause corresponding rotation of a corresponding actuator to radially compress the prosthetic valve.

Example 84. A prosthetic heart valve comprising:
a radially expandable and compressible frame comprising:
   a plurality of circumferentially spaced, axially extending posts;
   a plurality of circumferentially spaced, pairs of axially extending frame members, wherein each pair of frame members includes an axially extending proximal frame member and an axially extending distal frame member that is axially spaced from the proximal frame member to define a gap therebetween, wherein each pair of frame members is positioned circumferentially between two posts;
   a plurality of connecting struts coupling the posts to the proximal and distal frame members, wherein each post is connected to (i) an adjacent distal frame member of a first adjacent pair of frame members by two connecting struts, (ii) an adjacent proximal frame member of the first adjacent pair by two connecting struts, (iii) an adjacent distal frame member of a second adjacent pair of frame members by two connecting struts, and (iv) an adjacent proximal frame member of the second adjacent pair by two connecting struts, wherein each connecting strut has a concave curved portion and a convex curved portion separated by an inflection point;
   a plurality of actuators extending through respective pairs of first and second frame members, wherein the actuators are configured to radially expand the frame from a radially compressed state to a radially expanded state; and
a valvular structure disposed within the frame and configured to regulate the flow of blood through the frame in one direction.

Example 85. The prosthetic heart valve of any example herein, particularly example 84, wherein the struts, the posts, and the proximal and distal frame members are arranged to form a plurality of hexagonal cells and a plurality of diamond-shaped cells, wherein each diamond-shaped cell disposed within one of the hexagonal cells.

Example 86. The prosthetic heart valve of any example herein, particularly example 85, wherein each diamond-shaped cell is centered within a respective hexagonal cell.

Example 87. The prosthetic heart valve of any example herein, particularly any one of examples 85-86, wherein each hexagonal cell defines an inflow apex of the frame and an outflow apex of the frame.

Example 88. The prosthetic heart valve of any example herein, particularly any one of examples 85-87, wherein there are exactly six hexagonal cells and exactly six diamond-shaped cells.

Example 89. The prosthetic heart valve of any example herein, particularly any one of examples 85-88, wherein the hexagonal cells extend the entire length of the frame.

Example 90. The prosthetic heart valve of any example herein, particularly any one of examples 84-89, wherein the valvular structure comprises a plurality of leaflets, each leaflet comprising two commissure tabs on opposite sides of the leaflet, wherein each commissure tab is paired with an adjacent commissure tab of an adjacent leaflet to form a commissure, wherein each commissure is secured to an adjacent post.

Example 91. The prosthetic heart valve of any example herein, particularly example 90, wherein each post that is adjacent a commissure comprises a slot and a pair of commissure tabs of the adjacent commissure extends through the slot.

Example 92. The prosthetic heart valve of any example herein, particularly any one of examples 84-91, wherein the plurality of actuators comprise threaded rods having external threads.

Example 93. The prosthetic heart valve of any example herein, particularly example 92, wherein for each pair of proximal and distal frame members that have an actuator extending therethrough, the proximal frame member has a non-threaded bore, the distal frame member has a bore with internal threads, and the actuator extends through the non-threaded bore of the proximal frame member and the bore of the distal frame member, wherein the external threads of the actuator engage the internal threads of the distal frame member.

Example 94. The prosthetic heart valve of any example herein, particularly example 93, wherein for each pair of proximal and distal frame members that have an actuator extending therethrough, the distal frame member includes a cut-out region and a nut disposed in the cut-out region, wherein the nut defines the internal threads of the bore of the distal member.

Example 95. The prosthetic heart valve of any example herein, particularly any one of examples 84-94, wherein each post includes a cantilevered extension portion that extends toward an inflow end of the frame.

Example 96. The prosthetic heart valve of any example herein, particularly example 95, further comprising an inner skirt and/or an outer skirt mounted on the frame, wherein the inner skirt and/or the outer skirt are secured to the extension portions with sutures.

Example 97. The prosthetic heart valve of any example herein, particularly any one of examples 95-96, wherein cusp edge portions of leaflets of the valvular structure are secured to the extension portions with sutures.

Example 98. A prosthetic heart valve comprising:
a radially expandable and compressible frame comprising:
   a plurality of pairs of axially extending frame members, wherein each pair of frame members includes a first frame member and a second frame member axially spaced from the first frame member, wherein the pairs of frame members are circumferentially spaced from each other around a circumference of the frame, wherein the first frame member of each pair includes a cut-out region housing a nut having internal threads;
   a plurality of threaded rods extending through respective pairs of first and second frame members, wherein each threaded rod includes external threads that engage the internal threads of a corresponding nut, wherein rotation of the threaded rods in a first direction produces radial expansion of the frame from a radially compressed state to a radially expanded state;
a valvular structure disposed within the frame and configured to regulate the flow of blood through the frame in one direction.

Example 99. The prosthetic heart valve of any example herein, particularly example 98, wherein the frame further comprises a plurality of axially extending posts and a plurality of connecting struts, wherein each post is circumferentially disposed between a first and a second pair of frame members and the connecting struts couple the posts to the pairs of frame members.

Example 100. The prosthetic heart valve of any example herein, particularly example 99, wherein each post is connected to (i) an adjacent first frame member of a first adjacent pair of frame members by two connecting struts, (ii) an adjacent second frame member of the first adjacent pair by two connecting struts, (iii) an adjacent first frame member of a second adjacent pair of frame members by two connecting struts, and (iv) an adjacent second frame member of the second adjacent pair by two connecting struts.

Example 101. The prosthetic heart valve of any example herein, particularly any one of examples 99-100, wherein each connecting strut has a concave curved portion and a convex curved portion separated by an inflection point.

Example 102. The prosthetic heart valve of any example herein, particularly any one of examples 99-101, wherein the struts, the posts, and the first and second frame members are arranged to form a plurality of hexagonal cells and a plurality of diamond-shaped cells, wherein each diamond-shaped cell disposed within one of the hexagonal cells.

Example 103. The prosthetic heart valve of any example herein, particularly example 102, wherein each diamond-shaped cell is centered within a respective hexagonal cell.

Example 104. The prosthetic heart valve of any example herein, particularly any one of examples 102-103, wherein each hexagonal cell defines an inflow apex of the frame and an outflow apex of the frame.

Example 105. The prosthetic heart valve of any example herein, particularly any one of examples 102-104, wherein there are exactly six hexagonal cells and exactly six diamond-shaped cells.

Example 106. The prosthetic heart valve of any example herein, particularly any one of examples 102-105, wherein the hexagonal cells extend the entire length of the frame.

Example 107. The prosthetic heart valve of any example herein, particularly any one of examples 98-106, wherein the valvular structure comprises a plurality of leaflets, each leaflet comprising two commissure tabs on opposite sides of the leaflet, wherein each commissure tab is paired with an adjacent commissure tab of an adjacent leaflet to form a commissure, wherein each commissure is secured to an adjacent post.

Example 108. The prosthetic heart valve of any example herein, particularly example 107, wherein each post that is adjacent a commissure comprises a slot and a pair of commissure tabs of the adjacent commissure extends through the slot.

Example 109. A delivery apparatus, comprising:
one or more actuator assemblies extending from a handle, each actuator assembly comprising:
an outer sleeve;
an actuation member extending through the outer sleeve and having a distal end portion configured to releasably couple an actuator of a prosthetic heart valve, the distal end portion comprising:
   a driver head comprising an engagement member extending distally from a main body of the driver head, the engagement member configured to extend into a corresponding recess in a head portion of the actuator, and
   a gripper member comprising a plurality of arms configured to releasably couple the head portion of the actuator, the plurality of arms each comprising a distal end portion having a tooth extending radially inwardly toward a longitudinal axis of the actuation member and each arm having an arcuate sectional shape;
wherein rotation of the actuation member in a first direction radially expands the prosthetic heart valve and rotation of the actuation member in a second direction radially compresses the prosthetic heart valve.

Example 110. The delivery apparatus of any example herein, particularly example 109, wherein a distal end of the outer sleeve comprises first and second support extensions.

Example 111. The delivery apparatus of any example herein, particularly example 110, wherein the first support extension is configured to extend partially over a radially inner surface of the prosthetic valve and the second support extension is configured to extend partially over a radially outer surface of the prosthetic valve to inhibit rotation of the frame of the prosthetic valve relative to the actuator assemblies.

Example 112. The delivery apparatus of any example herein, particularly any one of examples 109-111, wherein the plurality of arms is movable between an expanded position and a compressed position.

Example 113. The delivery apparatus of any example herein, particularly example 112, wherein the plurality of arms is configured to bias radially outwardly away from a longitudinal axis of the actuation member.

Example 114. The delivery apparatus of any example herein, particularly any one of examples 109-113, wherein the engagement member comprises a square shape in cross-section.

Example 115. The delivery apparatus of any example herein, particularly any one of examples 109-114, wherein the engagement member comprises one or more facets configured to engage corresponding facets in the corresponding recess in the head portion of the actuator.

Example 116. The delivery apparatus of any example herein, particularly any one of examples 109-115, wherein the driver head comprises one or more projections extending radially outwardly from the driver head.

Example 117. The delivery apparatus of any example herein, particularly example 116, wherein when the plurality of arms is in a compressed position, the one or more projections are disposed between adjacent arms.

Example 118. The delivery apparatus of any example herein, particularly any one of examples 116-117, wherein the projections comprise a rectangular prism shape having chamfered side edges.

Example 119. The delivery apparatus of any example herein, particularly any one of examples 116-118, wherein the one or more projections comprise four projections spaced apart from one another about a perimeter of the driver head.

Example 120. The delivery apparatus of any example herein, particularly any one of examples 109-119, wherein the gripper member comprises an annular base member and the plurality of arms extend distally from the annular base member.

Example 121. The delivery apparatus of any example herein, particularly any one of examples 109-120, wherein the plurality of arms and the outer sleeve form a collet.

Example 122. The delivery apparatus of any example herein, particularly any one of examples 109-121, wherein each tooth comprises an angled proximal surface.

Example 123. The delivery apparatus of any example herein, particularly any one of examples 109-122, wherein a distal end portion of each arm flares radially outwardly such that the distal end portion is radially thicker than a main body of the arm.

Example 124. The delivery apparatus of any example herein, particularly any one of examples 109-123, wherein the plurality of arms comprises four arms.

Example 125. The delivery apparatus of any example herein, particularly any one of examples 109-124, wherein a distal end portion of each arm has an increased circumferential width relative to a main body of the arm.

Example 126. An assembly, comprising:
a prosthetic heart valve comprising:
   a radially expandable and compressible frame having an inflow end portion and an outflow end portion, and
   one or more actuators having a main body and a head portion, the actuators configured to radially expand the frame upon rotation of the one or more actuators, the head portion comprising a central recess and a circumferentially extending recess; and
a delivery apparatus comprising:
   a handle,
   one or more actuator assemblies extending from the handle, each actuator assembly comprising an outer sleeve and an actuation member extending through the outer sleeve, the actuation member having a distal end portion comprising:
   a driver head comprising an engagement member extending distally from a main body of the driver head that extends into the central recess of a respective actuator, and
   a gripper member comprising a plurality of arms configured to releasably couple the head portion of the respective actuator, the plurality of arms each comprising a distal end portion having a tooth that extends into the circumferential recess of the respective actuator; and
wherein rotation of the actuation member in a first direction radially expands the prosthetic heart valve and rotation of the actuation member in a second direction radially compresses the prosthetic heart valve.

Example 127. The assembly of any example herein, particularly example 126, wherein the central recess comprises a square shape in cross-section.

Example 128. The delivery apparatus of any example herein, particularly any one of examples 126-127, wherein the engagement member comprises a square shape in cross-section.

Example 129. The assembly of any example herein, particularly any one of examples 126-128, wherein the engagement member comprises one or more facets configured to engage corresponding facets in the central recess.

Example 130. The assembly of any example herein, particularly any one of examples 126-129, wherein the circumferential recess extends circumferentially around the head portion and comprises an angled proximal surface.

Example 131. The assembly of any example herein, particularly any one of examples 126-130, wherein each tooth comprises an angled proximal surface corresponding to the angled proximal surface of the circumferential recess.

Example 132. The assembly of any example herein, particularly any one of examples 126-131, wherein a distal end of the outer sleeve comprises first and second support extensions.

Example 133. The assembly of any example herein, particularly example 132, wherein the first support extension is configured to extend partially over a radially inner surface of the prosthetic valve and the second support extension is configured to extend partially over a radially outer surface of the prosthetic valve to inhibit rotation of the frame of the prosthetic valve relative to the actuator assemblies.

Example 134. The assembly of any example herein, particularly any one of examples 126-133, wherein the plurality of arms is movable between an expanded position and a compressed position.

Example 135. The assembly of any example herein, particularly example 134, wherein the plurality of arms is configured to bias radially outwardly away from a longitudinal axis of the actuation member.

Example 136. The assembly of any example herein, particularly any one of examples 126-135, wherein the driver head comprises one or more projections extending radially outwardly from the driver head.

Example 137. The assembly of any example herein, particularly example 136, wherein when the plurality of arms is in a compressed position, the one or more projections are disposed between adjacent arms.

Example 138. The assembly of any example herein, particularly any one of examples 136-137, wherein the projections comprise a rectangular prism shape having chamfered side edges.

Example 139. The assembly of any one of claims 136-138, wherein the one or more projections comprise four projections spaced apart from one another about a perimeter of the driver head.

Example 140. The assembly of any example herein, particularly any one of examples 126-139, wherein the gripper member comprises an annular base member and the plurality of arms extend distally from the annular base member.

Example 141. The assembly of any example herein, particularly any one of examples 126-140, wherein the plurality of arms and the outer sleeve form a collet.

Example 142. The assembly of any example herein, particularly any one of examples 126-141, wherein a distal end portion of each arm flares radially outwardly such that the distal end portion is radially thicker than a main body of the arm.

Example 143. The assembly of any example herein, particularly any one of examples 126-142 wherein the plurality of arms comprises four arms.

Example 144. The assembly of any example herein, particularly any one of examples 126-143, wherein a distal end portion of each arm has an increased circumferential width relative to a main body of the arm.

Example 145. The assembly of any example herein, particularly any one of examples 126-145, wherein the frame comprises a plurality of pairs of axially extending frame members, wherein each pair of frame members includes a first frame member and a second frame member axially spaced from the first frame member, and wherein the one or more actuators each extend through a respective pair of frame members.

Example 146. The assembly of any example herein, particularly example 145, wherein the pairs of frame members are circumferentially spaced from each other around a circumference of the frame.

Example 147. A method, comprising:
advancing an actuation member of an actuator assembly of a delivery apparatus axially relative to an actuator of a radially expandable and compressible prosthetic valve such that a plurality of arms of the actuation member advance over a head portion of the actuator, the head portion comprising a central recess and a circumferential recess;
continuing to advance the actuation member such that teeth extending radially inwardly from the plurality of arms are disposed within the circumferential recess and such that an engagement member extending distally from the actuation member is disposed within the central recess; and
advancing an outer sleeve of the actuator assembly over the actuation member such that the plurality of arms is radially compressed thereby retaining the teeth within the circumferential recess and coupling the actuator assembly to the prosthetic valve.

Example 148. A delivery apparatus, comprising:
one or more actuator assemblies extending from a handle, each actuator assembly comprising:
   an outer sleeve;
   an actuation member extending through the outer sleeve and having a distal end portion configured to releasably couple an actuator of a prosthetic heart valve, the distal end portion comprising:
      a driver head comprising an engagement member extending distally from a main body of the driver head, the engagement member configured to extend into a corresponding recess in a head portion of the actuator, the engagement member comprising one or more facets configured to engage corresponding facets in the corresponding recess, and
      a gripper member comprising a plurality of arms configured to releasably couple the head portion of the actuator, the plurality of arms each comprising a distal end portion having a tooth extending radially inwardly toward a longitudinal axis of the actuation member, the distal end portion of each arm having an increased circumferential width relative to a main body of the arm;
wherein rotation of the actuation member in a first direction radially expands the prosthetic valve and rotation of the actuation member in a second direction radially compresses the prosthetic valve.

Example 148. An implantable prosthetic device, comprising:
a radially expandable and compressible frame having an inflow end portion and an outflow end portion, the frame comprising:
   a plurality of posts, one or more of which are configured as actuation mechanisms comprising:
   a first frame member having a first inner bore,
   a second frame member having a second inner bore, the first and second frame members being spaced apart axially from one another, and
   an actuator having an external threaded surface and extending through the first and second inner bores, the actuator comprising a main body and a head portion, the head portion comprising a circumferential recess; and
   a plurality of struts coupling adjacent posts to one another; and
wherein rotation of the actuator in a first direction results in axial movement of the first and second frame members toward one another to radially expand the prosthetic device.

Example 149. The implantable prosthetic device of any example herein, particularly example 148, wherein the circumferential recess comprises an angled proximal surface.

Example 150. The implantable prosthetic device of any example herein, particularly any one of examples 148-149, wherein the head portion further comprises a central recess.

Example 151. The implantable prosthetic device of any example herein, particularly example 150, wherein the central recess comprises one or more facets configured to engage corresponding facets on an actuation assembly of a delivery apparatus.

Example 152. The implantable prosthetic device of any example herein, particularly any one of examples 150-151, wherein the central recess has a square shape in cross-section.

Example 153. A delivery apparatus, comprising:
one or more actuator assemblies extending from a handle, each actuator assembly comprising:
an outer sleeve including first and second support extensions extending from a distal end portion thereof;
an actuation member extending through the outer sleeve and having a distal engagement portion configured to releasably couple an actuator of a prosthetic heart valve, the distal engagement portion comprising:
   a driver head comprising an engagement element configured to engage a corresponding engagement element in a head portion of the actuator, and
   a gripper member comprising a plurality of arms configured to releasably couple the head portion of the actuator, the plurality of arms each comprising a first, proximal end portion and a second, distal end portion, the distal end portion having a circumferential width wider than a circumferential width of the proximal end portion and each arm having an arcuate sectional shape;
wherein rotation of the actuation member in a first direction radially expands the prosthetic heart valve and rotation of the actuation member in a second direction radially compresses the prosthetic heart valve.

Example 154. The delivery apparatus of any example herein, particularly example 153, wherein the driver head engagement element comprises a projection extending distally from a main body of the driver head, the projection having a square shape in cross-section.

Example 155. The delivery apparatus of any example herein, particularly example 154, wherein the engagement element of the head portion of the actuator comprises a corresponding recess, and wherein the driver head engagement element comprises one or more facets configured to engage corresponding facets in the corresponding recess.

Example 156. The delivery apparatus of any example herein, particularly any one of examples 153-155, wherein the driver head comprises one or more projections extending radially outwardly from the driver head such that when the plurality of arms is in a compressed position, the one or more projections are disposed between adjacent arms.

Example 157. The delivery apparatus of any example herein, particularly example 156, wherein the projections comprise a rectangular prism shape having chamfered side edges.

Example 158. The delivery apparatus of any example herein, particularly any one of examples 153-157, wherein the plurality of arms and the outer sleeve together form a collet.

Example 159. The delivery apparatus of any example herein, particularly any one of examples 153-158, wherein each arm comprises a tooth extending radially inwardly toward a longitudinal axis of the actuation member, and wherein each tooth comprises an angled proximal surface.

Example 160. The delivery apparatus of any example herein, particularly any one of examples 153-159, wherein a distal end portion of each arm flares radially outwardly such that the distal end portion is radially thicker than a main body of the arm.

Example 161. The delivery apparatus of any example herein, particularly example 153, wherein the driver head engagement element comprises a recess having a semi-circular shape in cross section.

Example 162. The delivery apparatus of any example herein, particularly example 161, wherein the wherein the engagement element of the head portion of the actuator comprises a corresponding Ω-shaped projection configured to be disposed at least partially within the recess.

Example 163. The delivery apparatus of any example herein, particularly any one of examples 161-162, wherein each arm has a uniform thickness from the proximal end portion to the distal end portion.

Example 164. The delivery apparatus of any example herein, particularly any one of examples 153-163, wherein a portion of the outer sleeve comprises a hypotube configured to be axially expandable and compressible between a first length and a second length.

Example 165. The delivery apparatus of any example herein, particularly example 164, wherein the hypotube comprises a plurality of diamond shaped slots that extend at least partially around a circumference of the outer sleeve.

Example 166. A delivery apparatus, comprising:
one or more actuator assemblies extending from a handle, each actuator assembly comprising:
an outer sleeve;
an actuation member extending through the outer sleeve and having a distal end portion configured to releasably couple an actuator of a prosthetic heart valve, the distal end portion comprising:
a driver head comprising an engagement member extending distally from a main body of the driver head, the engagement member configured to extend into a corresponding recess in a head portion of the actuator, the driver head comprising one or more projections extending radially from the driver head, and
a gripper member comprising a plurality of arms configured to releasably couple the head portion of the actuator, the plurality of arms each comprising a distal end portion having a tooth extending radially inwardly toward a longitudinal axis of the actuation member and each arm having an arcuate sectional shape;
wherein rotation of the actuation member in a first direction radially expands the prosthetic heart valve and rotation of the actuation member in a second direction radially compresses the prosthetic heart valve.

Example 167. The delivery apparatus of any example herein, particularly example 166, wherein a distal end of the outer sleeve comprises first and second support extensions and wherein the first support extension is configured to extend partially over a radially inner surface of the prosthetic valve and the second support extension is configured to extend partially over a radially outer surface of the prosthetic valve to inhibit rotation of the frame of the prosthetic valve relative to the actuator assemblies.

Example 168. The delivery apparatus of any example herein, particularly any one of examples 166-167, wherein when the plurality of arms is in a compressed position, the one or more projections are disposed between adjacent arms.

Example 169. A delivery apparatus, comprising:
one or more actuator assemblies extending from a handle, each actuator assembly comprising:
an outer sleeve;
an actuation member extending through the outer sleeve and having a distal end portion configured to releasably couple an actuator of a prosthetic heart valve, the distal end portion comprising:
   a driver head comprising an engagement recess extending proximally into a main body of the driver head, the engagement recess configured to accept a portion of a corresponding engagement member of a head portion of the actuator, the driver head comprising one or more shoulders extending radially from the driver head, each shoulder comprising a cutout configured to accept a portion of the corresponding engagement member, and
   a gripper member comprising a plurality of arms configured to releasably couple the head portion of the actuator, the plurality of arms each having an arcuate sectional shape, each pair of adjacent arms of the plurality of arms defining an Ω-shaped recess between them;
wherein rotation of the actuation member in a first direction radially expands the prosthetic heart valve and rotation of the actuation member in a second direction radially compresses the prosthetic heart valve.

Example 170. The delivery apparatus of any example herein, particularly example 169, wherein the engagement member of the head portion of the actuator comprises an Ω-shaped projection configured to be disposed at least partially within the engagement recess and within one or more of the Ω-shaped recesses of the gripper member.

Example 171. The delivery apparatus of any example herein, particularly any one of examples 169-170, wherein each arm has a uniform thickness from the proximal end portion to the distal end portion.

Example 172. The delivery apparatus of any example herein, particularly any one of examples 169-171, wherein each arm of the plurality of arms has a neck portion having a first circumferential width and a distal end portion having a second circumferential width greater than the first circumferential width.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A delivery apparatus, comprising:
one or more actuator assemblies extending from a handle, each actuator assembly comprising:
an outer sleeve including first and second support extensions extending from a distal end portion thereof; and
an actuation member extending through the outer sleeve and having a distal engagement portion configured to releasably couple an actuator of a prosthetic heart valve, the distal engagement portion comprising:
a driver head comprising an engagement element configured to engage a corresponding engagement element in a head portion of the actuator, and
a gripper member comprising a plurality of arms configured to releasably couple the head portion of the actuator, the plurality of arms each comprising a first, proximal end portion and a second, distal end portion, the distal end portion having a circumferential width wider than a circumferential width of the proximal end portion and each arm having an arcuate sectional shape;
wherein rotation of the actuation member in a first direction radially expands the prosthetic heart valve and rotation of the actuation member in a second direction radially compresses the prosthetic heart valve.

2. The delivery apparatus of claim 1, wherein the driver head engagement element comprises a projection extending distally from a main body of the driver head, the projection having a square shape in cross-section.

3. The delivery apparatus of claim 2, wherein the engagement element of the head portion of the actuator comprises a corresponding recess, and wherein the driver head engagement element comprises one or more facets configured to engage corresponding facets in the corresponding recess.

4. The delivery apparatus of claim 1, wherein the driver head comprises one or more projections extending radially outwardly from the driver head such that when the plurality of arms is in a compressed position, the one or more projections are disposed between adjacent arms.

5. The delivery apparatus of claim 4, wherein the projections comprise a rectangular prism shape having chamfered side edges.

6. The delivery apparatus of claim 1, wherein the plurality of arms and the outer sleeve together form a collet.

7. The delivery apparatus of claim 1, wherein each arm comprises a tooth extending radially inwardly toward a longitudinal axis of the actuation member, and wherein each tooth comprises an angled proximal surface.

8. The delivery apparatus of claim 1, wherein a distal end portion of each arm flares radially outwardly such that the distal end portion is radially thicker than a main body of the arm.

9. The delivery apparatus of claim 1, wherein the driver head engagement element comprises a recess having a semi-circular shape in cross section.

10. The delivery apparatus of claim 9, wherein the engagement element of the head portion of the actuator comprises a corresponding Ω-shaped projection configured to be disposed at least partially within the recess.

11. The delivery apparatus of claim 9, wherein each arm has a uniform thickness from the proximal end portion to the distal end portion.

12. The delivery apparatus of claim 1, wherein a portion of the outer sleeve comprises a hypotube configured to be axially expandable and compressible between a first length and a second length.

13. The delivery apparatus of claim 12, wherein the hypotube comprises a plurality of diamond shaped slots that extend at least partially around a circumference of the outer sleeve.

14. A delivery apparatus, comprising:
one or more actuator assemblies extending from a handle, each actuator assembly comprising:
an outer sleeve; and
an actuation member extending through the outer sleeve and having a distal end portion configured to releasably couple an actuator of a prosthetic heart valve, the distal end portion comprising:
a driver head comprising an engagement member extending distally from a main body of the driver head, the engagement member configured to extend into a corresponding recess in a head portion of the actuator, the driver head comprising one or more projections extending radially from the driver head, and
a gripper member comprising a plurality of arms configured to releasably couple the head portion of the actuator, the plurality of arms each comprising a distal end portion having a tooth extending radially inwardly toward a longitudinal axis of the actuation member and each arm having an arcuate sectional shape;
wherein rotation of the actuation member in a first direction radially expands the prosthetic heart valve and rotation of the actuation member in a second direction radially compresses the prosthetic heart valve.

15. The delivery apparatus of claim 14, wherein a distal end of the outer sleeve comprises first and second support extensions and wherein the first support extension is configured to extend partially over a radially inner surface of the prosthetic valve and the second support extension is configured to extend partially over a radially outer surface of the prosthetic valve to inhibit rotation of the prosthetic valve relative to the actuator assemblies.

16. The delivery apparatus of claim 14, wherein when the plurality of arms is in a compressed position, the one or more projections are disposed between adjacent arms.

17. A delivery apparatus, comprising:
one or more actuator assemblies extending from a handle, each actuator assembly comprising:
an outer sleeve; and
an actuation member extending through the outer sleeve and having a distal end portion configured to releasably couple an actuator of a prosthetic heart valve, the distal end portion comprising:
a driver head comprising an engagement recess extending proximally into a main body of the driver head, the engagement recess configured to accept a portion of a corresponding engagement member of a head portion of the actuator, the driver head comprising one or more shoulders extending radially from the driver head, each shoulder comprising a cutout configured to accept a portion of the corresponding engagement member, and
a gripper member comprising a plurality of arms configured to releasably couple the head portion of the actuator, the plurality of arms each having an arcuate sectional shape, each pair of adjacent arms of the plurality of arms defining an Ω-shaped recess between them;
wherein rotation of the actuation member in a first direction radially expands the prosthetic heart valve and rotation of the actuation member in a second direction radially compresses the prosthetic heart valve.

18. The delivery apparatus of claim 17, wherein the engagement member of the head portion of the actuator comprises an Ω-shaped projection configured to be disposed at least partially within the engagement recess and within one or more of the Ω-shaped recesses of the gripper member.

19. The delivery apparatus of claim 17, wherein each arm has a uniform thickness from a proximal end portion to a distal end portion.

20. The delivery apparatus of claim 17, wherein each arm of the plurality of arms has a neck portion having a first circumferential width and a distal end portion having a second circumferential width greater than the first circumferential width.
